(19) United States
(12) Patent Application Publication
(10) Pub. No.: US 2023/0338586 A1
(43) Pub. Date: Oct. 26, 2023

(54) MULTIFUNCTIONAL CONTRAST AGENTS, COMPOSITIONS, AND METHODS

(71) Applicants: The Florida State University Research Foundation, Inc., Tallahassee, FL (US); Laboratory of Coordination Chemistry of the French National Centre for Scientific Research, Toulouse (FR)

(72) Inventors: Hedi Mattoussi, Tallahassee, FL (US); Liang Du, Tallahassee, FL (US); Wentao Wang, Tallahassee, FL (US); Catherine Amiens, Toulouse (FR); Samuel Grant, Tallahassee, FL (US)

(21) Appl. No.: 18/305,553

(22) Filed: Apr. 24, 2023

Related U.S. Application Data
(60) Provisional application No. 63/333,909, filed on Apr. 22, 2022.

(51) Int. Cl. A61K 49/12

(52) U.S. Cl. CPC A61K 49/126

(57) ABSTRACT

Contrast agents, compositions including contrast agents, methods of forming contrast agents, and methods of imaging. Contrast agents may have a desirable solubility in liquids, such as aqueous liquids. Methods of forming contrast agents may include contacting a polymer that includes anhydride functional groups with one or more compounds. Methods of imaging may include administering to patients a contrast agent or composition including a contrast agent.

### CROSS-REFERENCE TO RELATED APPLICATIONS

This application claims priority to U.S. Provisional Patent Application No. 63/333,909, filed Apr. 22, 2022, which is incorporated herein by reference.

### STATEMENT REGARDING FEDERALLY SPONSORED RESEARCH OR DEVELOPMENT

This invention was made with government support under Contract Nos. 150850 and 2005079, awarded by the National Science Foundation, and Contract No. FA9550-18-1-0144, awarded by the Air Force Office of Scientific Research. The government has certain rights in this invention.

### BACKGROUND

Magnetic resonance imaging (MRI) is a noninvasive technique with a wide range of applications in biology, medicine, and materials science. For instance, ¹H-based MRI can permit the noninvasive visualization of soft and deep tissue, by exploiting the combination of high natural abundance and large gyromagnetic ratio of protons, to allow the acquisition of images with good spatial resolution (sub-millimeter).

¹H-based MRI has been used over the decades to acquire a large amount of data that has consolidated its clinical values in diagnostics and biomedicine. It has also been shown that MRI provides highly informative data that can help in neuroscience and biomedicine, with particularly strong clinical applications in tumor diagnosis and localization. ¹H-based MRI generally relies on changes in the transverse (T₂) and longitudinal (T₁) spin relaxation properties of water protons distributed in living tissues. It is often employed in combination with a specially-designed contrast agent (CA), which is believed to shorten the relaxation of proximal proton spins and enhance the image contrast and resolution. CAs, such as those made from Gd(III) paramagnetic complexes and superparamagnetic iron oxide (SPIO) nanoparticles, are widely used in proton-based MRI, but they still face one or more practical limitations. These CAs, respectively, can affect the longitudinal (T₁) or transverse (T₂) relaxation times of surrounding tissue(s), and their use typically requires pre- and post-injection imaging, which can result in long scan times, while frequently yielding large background signals (see, e.g., Shan, L. et al. J. Nanopart. Res. 2012, 14, 1122). Moreover, relatively high doses of the agent usually must be administered to increase the contrast, which can induce toxicity to tissue and organs (see, e.g., Tirotta, I. et al. Chem. Rev. 2015, 115, 1106-1129).

To circumvent these issues, contrast agents exploiting other nuclei have been developed and tested as direct labels in MR imaging. Among those, ¹⁹F-based MRI has attracted much attention due to a few unique characteristics. ¹⁹F nuclei can exhibit high gyromagnetic ratio (comparable to that of protons), yielding pronounced NMR signal, and it has 100% natural abundance. Furthermore, due to the very low traces of intrinsic fluorine in biological tissues and organs, fluorine-rich tracers can be easily visualized in-vivo using ¹⁹F-based MRI, and provide bright signal compared to their surroundings (i.e., positive CAs) (see, e.g., Bulte, J. W. M. et al. Nat Biotechnol 2005, 23, 945-946; Kislukhin, A. A. et al. Nat. Mater. 2016, 15, 662-668; and Srinivas, M. et al. Magn. Reson. Med. 2009, 62, 747-753). It has been reported that because C—F bonds involve π-π_{F}-interactions they exhibit high enthalpy, which can impart them with a stabilizing energy up to ˜25 kJ/mol (Bacchi, S. et al. Chem. Eur. J. 2006, 12, 3538-3546).

This can make fluorine-rich molecules chemically and thermally more stable in biological media compared to their hydrocarbon counterparts; this has led to the development of novel plastics, surfactants, and pharmaceuticals (see, e.g., Berger, R. et al. Chem. Soc. Rev. 2011, 40, 3496-3508). Early work indicated the feasibility of ¹⁹F imaging using sodium fluoride and perfluorotributylamine (Holland, G. N. et al. J. Magn. Reson. (1969) 1977, 28, 133-136). Subsequently, ¹⁹F-based MR has been actively investigated (see, e.g., Srinivas, M. et al. Magn. Reson. Med. 2009, 62, 747-753). The low trace amount of intrinsic signal in biological systems presents a unique advantage which has been exploited, for example, to realize quantitative cell trafficking and physiology assessments (see, e.g., Chen, J. et al. Wiley Interdisciplinary Reviews: Nanomedicine and Nanobiotechnology 2010, 2, 431-440). In addition, ¹⁹F NMR signatures cover a wide range of chemical shifts which can be sensitive to pH changes and oxygen concentration (i.e., oxygen tension, pO₂) (see, e.g., Mehta, V. D. et al. Bioconjugate Chem. 1994, 5, 257-261; Nöth, U. et al. Int. J. Radiat. Oncol. Biol. Phys. 2004, 60, 909-919). Transition-metal complexes containing ¹⁹F have also been developed as MR pH sensors (Gaudette, A. I. et al. Chem. Commun. 2017, 53, 12962-12965).

Perfluorocarbons (PFCs) have been widely investigated for designing ¹⁹F CAs. For example, hexafluorobenzene (HFB) was used to map the oxygen tension in rabbit breast tumors (see, e.g., Xia, M. et al. Physics in Medicine and Biology 2005, 51, 45-60). Perfluorooctyl bromide (PFOB) and its derivatives were reported to be inert and safe for use in visualizing gastrointestinal tract (see, e.g., Mattrey, R. F. Radiology 1994, 191, 841-848). Perfluorinated crown ether (PFCE) and perfluoropolyether (PFPE) emulsions are currently state-of-the-art candidates for use in biological and preclinical studies (see, e.g., Kislukhin, A. A. et al. Nat. Mater. 2016, 15, 662-668).

However, one of the drawbacks of these PFC-based tracers is their very limited solubility in water. They tend to aggregate in biological environments, which can result in long retention times with associated toxicity (Zhang, C. et al. ACS Nano 2018, 12, 9162-9176). To address these problems, spatially-structured CAs have been designed, including micelle structures formed using amphiphilic molecules that present fluorine-rich hydrophobic tails, or poly(acrylic acid)-containing fluorinated acrylate (or methacrylate) moieties (see, e.g., Peng, H. et al. Biomacromolecules 2009, 10, 374-381). Dendritic polyamidoamine (PAMAM) macromolecules prepared with large numbers of ¹⁹F atoms have been tested for encapsulating drugs, while allowing controlled release (see, e.g., Criscione, J. M. et al. Biomaterials 2009, 30, 3946-3955). In addition, a range of partially fluorinated polymers (PFPs) have been explored as ¹⁹F-based CAs (see, e.g., Wang, K. et al. Macromol. Chem. Phys. 2016, 217, 2262-2274; Yu, W. et al. The Journal of Organic Chemistry 2015, 80, 4443-4449; Thurecht, K. J. et al. J. Am. Chem. Soc. 2010, 132, 5336-5337; Du, W. et al. Biomacromolecules 2008, 9, 2826-2833). Nonetheless, the high fluorine content (up to ˜30 wt %.) in these tracers has often resulted in serious solubility issues in biological conditions, implying that developing ¹⁹F CAs with high signal-to-noise ratio and better affinity to water is still as needed as ever.

There remains a need for CAs, including ¹⁹F CAs, that have improved solubility in certain liquids, such as aqueous liquids.

### BRIEF SUMMARY

Provided herein are methods of synthesizing fluorine-rich polymers, and fluorine-rich polymers that may be used as ¹⁹F CAs, such as CAs with a single NMR signature. Embodiments of the fluorine-rich polymers exhibit large signal-to-noise ratios, and/or tunable T₂ (and T₁) relaxation times, without undesirably compromising affinity to certain liquids, such as aqueous liquids. Embodiments of the methods described herein allow the introduction of a high concentration of ¹⁹F atoms per chain, while promoting high affinity to water. In some embodiments, through discreet manipulation of the macromolecular architecture, additional dynamic segmental mobility of the fluorinated moieties is introduced, which may result in a sizable increase in the relaxation time, coupled with narrowing of the generated ¹⁹F NMR peak, along with a significant enhancement in the measured intensity. In some embodiments, ¹H NMR measurements are used to deduce accurate estimates of the PEG-to-fluorine moieties in a given polymer.

In one aspect, contrast agents are provided. In some embodiments, the contrast agents are of the following formula:

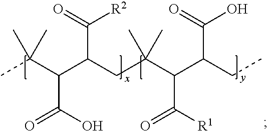

wherein a ratio of x:y is about 30:70 to about 99:1; wherein R¹ is a hydrocarbyl that includes an amine substituent, and one or more carbon atoms substituted with one or more fluorine atoms; and wherein R² includes a polar (e.g., water soluble) polymeric moiety, such as polyethylene glycol. The amine substituent of R¹ may be covalently bonded to the carbon atom of the carbonyl moiety to which “R¹” is bonded. R², in some embodiments, also includes at least one amine moiety, such as a secondary amine that is bonded to a monomer of the polyethylene glycol (or other polar polymeric moiety) and the carbon atom of the carbonyl to which R² is bonded. The contrast agents may have a solubility in an aqueous liquid of at least 1 mg/mL, or at least 10 mg/mL.

In another aspect, compositions are provided, which may include any one or more of the contrast agents provided herein. In some embodiments, the compositions include a liquid, such as an aqueous liquid, and one or more of the contrast agents described herein. In some embodiments, a concentration of the contrast agent in the liquid is about 10 mg/mL to about 250 mg/mL. The contrast agents may have a solubility in the liquid, such as an aqueous liquid, of at least 1 mg/mL, or at least 10 mg/mL.

In a further aspect, methods of imaging are provided. In some embodiments, the methods include administering to a patient one or more contrast agents and/or compositions described herein, and collecting an image of at least a portion of the patient with magnetic resonance imaging.

In a still further aspect, methods of forming contrast agents are provided. In some embodiments, the methods include providing a polymer formed of at least one type of monomer, wherein the at least one type of monomer includes a monomer including an anhydride; contacting the polymer with (a) a first compound including a primary amine and one or more carbon atoms substituted with one or more fluorine atoms, and (b) a second compound including polyethylene glycol substituted with a primary amine to form the contrast agent.

Additional aspects will be set forth in part in the description which follows, and in part will be obvious from the description, or may be learned by practice of the aspects described herein. The advantages described herein may be realized and attained by means of the elements and combinations particularly pointed out in the appended claims. It is to be understood that both the foregoing general description and the following detailed description are exemplary and explanatory only and are not restrictive.

### BRIEF SUMMARY OF THE DRAWINGS

FIG. 1A is a schematic of an embodiment of a nucleophilic addition reaction used to prepare an embodiment of a multi-functional polymer ligand.

FIG. 1B is a ¹⁹F nuclear magnetic resonance (NMR) spectrum.

FIG. 1C is a ¹⁹F nuclear magnetic resonance (NMR) spectrum.

FIG. 1D is a stacked ¹H NMR spectra of embodiments of multi-functional polymer ligands.

FIG. 2 depicts a ¹H NMR spectra of an embodiment of a multi-functional polymer ligand.

FIG. 3 depicts a ¹H NMR spectra of an embodiment of a multi-functional polymer ligand.

FIG. 4A is a schematic of an embodiment of an addition reaction used to prepare an embodiment of a multi-functional polymer ligand.

FIG. 4B is a ¹⁹F nuclear magnetic resonance (NMR) spectrum.

FIG. 4C is a ¹⁹F nuclear magnetic resonance (NMR) spectrum.

FIG. 4D is a stacked ¹⁹F NMR spectra of embodiments of multi-functional polymer ligands.

FIG. 5A depicts normalized integrated intensity v. echo times.

FIG. 5B depicts T₂ relaxation time v. molecular weight of embodiments of precursors.

FIG. 5C depicts a diffusion ordered spectroscopy (DOSY) spectrum of ˜10 mg/mL of an embodiment of a multi-functional polymer ligand.

FIG. 5D depicts a DOSY spectra of ˜10 mg/mL of an embodiment of a multi-functional polymer ligand.

FIG. 6A depicts a plot of normalized area of a spectra v. different echo times collected for an embodiment of a contrast agent.

FIG. 6B depicts a plot of normalized area of a spectra v. different echo times collected for an embodiment of a contrast agent.

FIG. 6C depicts a plot of normalized area of a spectra v. different echo times collected for an embodiment of a contrast agent.

FIG. 6D depicts a plot of normalized area of a spectra v. different echo times collected for an embodiment of a contrast agent.

FIG. 7A depicts a 2-D DOSY NMR spectra of intensity v. magnetic field gradient, collected for an embodiment of a multi-functional polymer ligand.

FIG. 7B depicts a 2-D DOSY NMR spectra of intensity v. magnetic field gradient, collected for an embodiment of a multi-functional polymer ligand.

FIG. 8A depicts signal intensity v. echo time for PEG-OMe-PIMA-PEG₆₀₀-CF₃ at a concentration of 200 mg/mL (PEG=polyethylene glycol; PIMA=poly(isobutylene-alt-maleic anhydride)).

FIG. 8B depicts signal intensity v. echo time for PEG-OMe-PIMA-CF₃ at a concentration of 200 mg/mL.

FIG. 8C depicts signal intensity v. echo time for PEG-OMe-PIMA-PEG₆₀₀-CF₃ at a concentration of 150 mg/mL.

FIG. 8D depicts signal intensity v. echo time for PEG-OMe-PIMA-CF₃ at a concentration of 150 mg/mL.

FIG. 8E depicts signal intensity v. echo time for PEG-OMe-PIMA-PEG₆₀₀-CF₃ at a concentration of 100 mg/mL.

FIG. 8F depicts signal intensity v. echo time for PEG-OMe-PIMA-CF₃ at a concentration of 100 mg/mL.

FIG. 8G depicts signal intensity v. echo time for PEG-OMe-PIMA-PEG₆₀₀-CF₃ at a concentration of 50 mg/mL.

FIG. 8H depicts signal intensity v. echo time for PEG-OMe-PIMA-CF₃ at a concentration of 50 mg/mL.

FIG. 9A depicts signal intensity v. echo time for PEG-OMe-PIMA-PEG₆₀₀-CF₃ at a concentration of 200 mg/mL.

FIG. 9B depicts signal intensity v. echo time for PEG-OMe-PIMA-CF₃ at a concentration of 200 mg/mL.

FIG. 9C depicts signal intensity v. echo time for PEG-OMe-PIMA-PEG₆₀₀-CF₃ at a concentration of 150 mg/mL.

FIG. 9D depicts signal intensity v. echo time for PEG-OMe-PIMA-CF₃ at a concentration of 150 mg/mL.

FIG. 9E depicts signal intensity v. echo time for PEG-OMe-PIMA-PEG₆₀₀-CF₃ at a concentration of 100 mg/mL.

FIG. 9F depicts signal intensity v. echo time for PEG-OMe-PIMA-CF₃ at a concentration of 100 mg/mL.

FIG. 9G depicts signal intensity v. echo time for PEG-OMe-PIMA-PEG₆₀₀-CF₃ at a concentration of 50 mg/mL.

FIG. 9H depicts signal intensity v. echo time for PEG-OMe-PIMA-CF₃ at a concentration of 50 mg/mL.

### DETAILED DESCRIPTION

Compounds, including polymeric compounds, are provided herein, which may be used as contrast agents. In some embodiments, the compounds include a structure of the following formula:

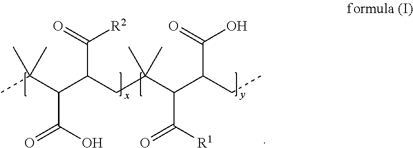

Formula (I) may include any ratio of x:y. In some embodiments, a ratio of x:y is about 1:99 to about 99:1, about 10:90 to about 99:1, about 20:80 to about 99:1, about 30:70 to about 99:1, about 40:60 to about 99:1, about 50:50 to about 99:1, about 60:40 to about 99:1, about 70:30 to about 99:1, about 80:20 to about 99:1, about 1:99 to about 90:10, about 10:90 to about 90:10, about 20:80 to about 90:10, about 30:70 to about 90:10, about 40:60 to about 90:10, about 50:50 to about 90:10, about 60:40 to about 90:10, about 70:30 to about 90:10, or about 80:20 to about 90:10.

Formula (I) may include (i) a random arrangement of R¹ and R² substituents along the backbone of the structure, (ii) one or more blocks of monomers that include (e.g., 10 or more consecutive monomers substituted with R¹ or R²) R¹ and/or R² substituents, or a combination thereof.

Within formula (I), R¹ may be any hydrocarbyl, such as a hydrocarbyl that does not undesirably impact the compounds' performance as a contrast agent. R¹ may include any number of carbon atoms; for example, in some embodiments, R¹ is a C₁-C₆₀ hydrocarbyl, a C₁₀-C₆₀ hydrocarbyl, a C₂₀-C₆₀ hydrocarbyl, or a C₄₀-C₆₀ hydrocarbyl. In some embodiments R¹ is a hydrocarbyl that is substituted with at least one amine. The at least one amine may be a tertiary amine, a secondary amine, a primary amine, or a combination thereof. In some embodiments, R 1 includes one or more carbon atoms substituted with one or more halogen atoms, such as fluorine atoms. For example, R¹ may include a carbon atom substituted with one, two, or three fluorine atoms (e.g., a trifluoromethyl group). An R¹ hydrocarbyl may be a polymeric hydrocarbyl, or at least a portion of its structure may be polymeric (i.e., include at least two monomers bonded together).

In some embodiments, R¹ is—

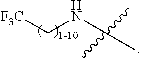

In some embodiments, R¹ is—

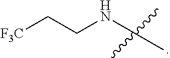 [SMILES: *NCCC(F)(F)F]

In some embodiments, R¹ is—

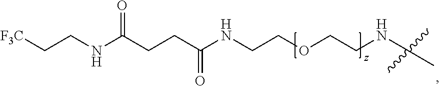

wherein z is 2 to 20, 4 to 20, 6 to 20, 8 to 20, 10 to 20, 12 to 20, 14 to 20, 16 to 20, 2 to 18, 2 to 16, 2 to 14, 2 to 12, 2 to 10, 2 to 8, 2 to 6, or 2 to 4.

Within formula (I), R² may be any hydrocarbyl, such as a hydrocarbyl that does not undesirably impact the compounds' performance as a contrast agent. R² may be a water-soluble hydrocarbyl; therefore, the hydrocarbyl of R² may be substituted with one or more polar substituents, as described herein. R² may include any number of carbon atoms; for example, in some embodiments, R² is a C₁-C₆₀ hydrocarbyl, a C₁₀-C₆₀ hydrocarbyl, a C₂₀-C₆₀ hydrocarbyl, or a C₄₀-C₆₀ hydrocarbyl. An R² hydrocarbyl may be a polymeric hydrocarbyl, or at least a portion of its structure may be polymeric (i.e., include at least two monomers bonded together). In some embodiments, R² includes polyethylene glycol. In some embodiments, R² further includes at least one amine, which may include a primary amine, a secondary amine, a tertiary amine, or a combination thereof. In some embodiments, the amine, such as a secondary amine, is bonded to at least one monomer of polyethylene glycol.

In some embodiments, R² is—

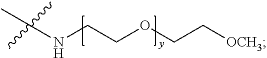

wherein v is 2 to 20.

### Compositions

Also provided herein are compositions that include one or more of the compounds provided herein. In some embodiments, the compositions include a liquid, and any one or more of the contrast agents disclosed herein.

In some embodiments, the liquid is an aqueous liquid.

The compositions provided herein may be at least slightly soluble in the liquid. In some embodiments, the compositions have a solubility in the liquid of at least 0.03 mg/mL, 0.06 mg/mL, 0.1 mg/mL, 0.5 mg/mL, 1 mg/mL, 10 mg/mL, 50 mg/mL, 100 mg/mL, 150 mg/mL, 200 mg/mL, or 250 mg/mL.

In some embodiments, a concentration of the contrast agent in the liquid is 0.03 mg/mL to about 250 mg/mL, 0.06 mg/mL to about 250 mg/mL, 0.1 mg/mL to about 250 mg/mL, 0.5 mg/mL to about 250 mg/mL, 1 mg/mL to about 250 mg/mL, 10 mg/mL to about 250 mg/mL, 50 mg/mL to about 250 mg/mL, 100 mg/mL to about 250 mg/mL, 150 mg/mL to about 250 mg/mL, or 200 mg/mL to about 250 mg/mL.

### Methods of Imaging

Also provided herein are methods of imaging, which may include the use of a composition or compound herein as a contrast agent.

In some embodiments, the methods include administering to a patient a contrast agent or a composition as described herein; and collecting an image of at least a portion of the patient with any known technique, such as magnetic resonance imaging (MRI). The patient may include any mammal, such as a human.

The contrast agent or composition may be configured for any suitable route of administration to the patient. The route may be selected to achieve local, regional or systemic delivery, as appropriate for the diagnostic imaging. The contrast agent or composition may be configured, for example, for oral or intravascular administration.

### Methods of Forming a Compound

Provided herein are methods of forming a compound, such as a contrast agent. In some embodiments, the methods include providing a polymer formed of at least one type of monomer, wherein the at least one type of monomer comprises a monomer comprising an anhydride. The polymer may be a homopolymer or copolymer.

In some embodiments, the polymer is formed by polymerizing maleic anhydride and at least one other comonomer, the comonomer including a polymerizable double bond. For example, the polymer may be a poly(alkylene-maleic anhydride), such as poly(isobutylene-alt-maleic anhydride). In some embodiments, the polymer is of the following formula:

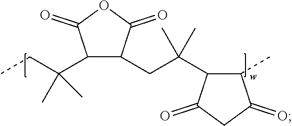

wherein w is 2 to 50, 10 to 40, 10 to 30, or 15 to 25.

In some embodiments, the methods also include contacting the polymer with (a) a first compound that includes a primary amine and one or more carbon atoms substituted with one or more fluorine atoms, and (b) a second compound including polyethylene glycol substituted with a primary amine to form the contrast agent.

The first compound may be any hydrocarbon that is capable of forming an R¹ substituent of formula (I) upon contacting an anhydride-containing polymer. The hydrocarbon include any number of carbon atoms; for example, in some embodiments, the first compound is a C₁-C₆₀ hydrocarbon, a C₁₀-C₆₀ hydrocarbon, a C₂₀-C₆₀ hydrocarbon, or a C₄₀-C₆₀ hydrocarbon. In some embodiments the first compound is a hydrocarbon that is substituted with at least one amine. The at least one amine may be a tertiary amine, a secondary amine, a primary amine, or a combination thereof. In some embodiments, the first compound includes one or more carbon atoms substituted with one or more halogen atoms, such as fluorine atoms. For example, the first compound may include a carbon atom substituted with one, two, or three fluorine atoms (e.g., a trifluoromethyl group). The first compound may be a polymeric hydrocarbon, or at least a portion of its structure may be polymeric (i.e., include at least two monomers bonded together). In some embodiments, the first compound is of the following formula:

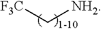

In some embodiments, the first compound is 3,3,3-trifluoropropan-1-amine. In some embodiments, the first compound is of the following formula:

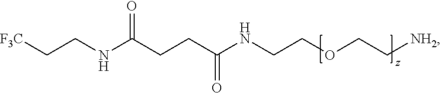

wherein z is 2 to 50, 2 to 40, 2 to 30, 2 to 20, 2 to 10, 10 to 20, 10 to 15, or 12.

The second compound may be any hydrocarbon capable of forming an R² substituent of formula (I) upon contacting an anhydride-containing polymer. The second hydrocarbon may be a water-soluble hydrocarbon; therefore, the hydrocarbon may be substituted with one or more polar substituents, as described herein. The second compound may be a hydrocarbon that includes any number of carbon atoms; for example, in some embodiments, the second compound is a C₁-C₆₀ hydrocarbon, a C₁₀-C₆₀ hydrocarbon, a C₂₀-C₆₀ hydrocarbon, or a C₄₀-C₆₀ hydrocarbon. The second compound may be a polymeric hydrocarbon, or at least a portion of its structure may be polymeric (i.e., include at least two monomers bonded together). In some embodiments, the second compound includes polyethylene glycol. In some embodiments, the second compound also includes at least one amine, which may include a primary amine, a secondary amine, a tertiary amine, or a combination thereof. In some embodiments, the amine, such as a secondary amine, is bonded to at least one monomer of polyethylene glycol. The primary amine of the second compound may be a terminal primary amine. The second compound may include a terminal alkoxy moiety, such as a terminal alkoxy moiety is a terminal methoxy moiety.

In some embodiments, the second compound is of the following formula:

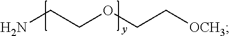

wherein y is 2 to 50, 2 to 40, 2 to 30, 2 to 20, 10 to 20, or 13 to 17.

In some embodiments, the polymer is contacted with a mole ratio of the first compound to the second compound of about 1:99 to about 99:1, about 10:90 to about 99:1, about 20:80 to about 99:1, about 30:70 to about 99:1, about 40:60 to about 99:1, about 50:50 to about 99:1, about 60:40 to about 99:1, about 70:30 to about 99:1, about 80:20 to about 99:1, about 1:99 to about 90:10, about 10:90 to about 90:10, about 20:80 to about 90:10, about 30:70 to about 90:10, about 40:60 to about 90:10, about 50:50 to about 90:10, about 60:40 to about 90:10, about 70:30 to about 90:10, about 80:20 to about 90:10, about 10:90 to about 50:50, or about 30:70 to about 50:50.

When used herein with regard to the selection of a substituent, the term “independently” indicates that (i) a substituent at a particular location may be the same or different for each molecule or monomer of a formula (e.g., a polymer may include two repeat units, with each repeat unit having the same or a different hydrocarbyl selected for R¹), and/or (ii) two differently labeled substituents selected from the same pool of substituents may be the same or different.

The term “hydrocarbyl”, as used herein, generally refers to aliphatic, aryl, or arylalkyl groups, including substituted derivatives thereof, as defined herein; and the term “hydrocarbon” refers to compounds capable of forming the “hydrocarbyl” substituents upon contacting a polymer as described herein. Examples of aliphatic groups, in each instance, include, but are not limited to, an alkyl group, a cycloalkyl group, an alkenyl group, a cycloalkenyl group, an alkynyl group, an alkadienyl group, a cyclic group, and the like, and includes all substituted, unsubstituted, branched, and linear analogs or derivatives thereof. Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl. Examples of aryl or arylalkyl moieties include, but are not limited to, anthracenyl, azulenyl, biphenyl, fluorenyl, indan, indenyl, naphthyl, phenanthrenyl, phenyl, 1,2,3,4-tetrahydro-naphthalene, tolyl, xylyl, mesityl, benzyl, and the like, including any heteroatom substituted derivative thereof.

Unless otherwise indicated, the term “substituted,” when used to describe a chemical structure or moiety, refers to a derivative of that structure or moiety wherein (i) a multi-valent non-carbon atom (e.g., oxygen, nitrogen, sulfur, phosphorus, etc.) is bonded to one or more carbon atoms of the chemical structure or moiety (e.g., a “substituted” C₄ hydrocarbyl may include, but is not limited to, diethyl ether moiety, a methyl propionate moiety, an N,N-dimethylacetamide moiety, a butoxy moiety, etc., and a “substituted” aryl C₁₂ hydrocarbyl may include, but is not limited to, an oxydibenzene moiety, a benzophenone moiety, etc.) or (ii) one or more of its hydrogen atoms (e.g., chlorobenzene may be characterized generally as an aryl C₆ hydrocarbyl “substituted” with a chlorine atom) is substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), tertiary amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, oxo, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-).

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

While certain aspects of conventional technologies have been discussed to facilitate disclosure of various embodiments, applicants in no way disclaim these technical aspects, and it is contemplated that the present disclosure may encompass one or more of the conventional technical aspects discussed herein.

The present disclosure may address one or more of the problems and deficiencies of known methods and processes. However, it is contemplated that various embodiments may prove useful in addressing other problems and deficiencies in a number of technical areas. Therefore, the present disclosure should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

In the descriptions provided herein, the terms “includes,” “is,” “containing,” “having,” and “comprises” are used in an open-ended fashion, and thus should be interpreted to mean “including, but not limited to.” When compositions or methods are claimed or described in terms of “comprising” various steps or components, the compositions or methods can also “consist essentially of” or “consist of” the various steps or components, unless stated otherwise.

The terms “a,” “an,” and “the” are intended to include plural alternatives, e.g., at least one. For instance, the disclosure of “a polymer”, “a liquid”, and the like, is meant to encompass one, or mixtures or combinations of more than one polymer, liquid, and the like, unless otherwise specified.

Various numerical ranges may be disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. Moreover, all numerical end points of ranges disclosed herein are approximate. As a representative example, Applicant discloses, in some embodiments, that z is 8 to 14. This range should be interpreted as encompassing 8 and 14, and further encompasses each of 9, 10, 11, 12, and 13, including any ranges and sub-ranges between any of these values.

As used herein, the term “about” means plus or minus 10% of the numerical value of the number with which it is being used.

### LISTING OF EMBODIMENTS

The following is a non-limiting listing of embodiments of the disclosure:
Embodiment 1. A contrast agent of the following formula:

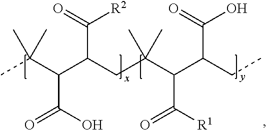

wherein a ratio of x:y is about 1:99 to about 99:1; wherein, independently, each R¹ is a hydrocarbyl that (i) is substituted with an amine, and (ii) comprises one or more carbon atoms substituted with one or more fluorine atoms; and wherein R² comprises polyethylene glycol.
Embodiment 2. The contrast agent of Embodiment 1, wherein the ratio of x:y is about 10:90 to about 99:1, about 20:80 to about 99:1, about 30:70 to about 99:1, about 40:60 to about 99:1, about 50:50 to about 99:1, about 60:40 to about 99:1, about 70:30 to about 99:1, about 80:20 to about 99:1, about 1:99 to about 90:10, about 10:90 to about 90:10, about 20:80 to about 90:10, about 30:70 to about 90:10, about 40:60 to about 90:10, about 50:50 to about 90:10, about 60:40 to about 90:10, about 70:30 to about 90:10, or about 80:20 to about 90:10.
Embodiment 3. The contrast agent of any of the preceding embodiments, wherein the hydrocarbyl of R¹ is a polymeric hydrocarbyl, or at least a portion of its structure is polymeric (i.e., includes at least two monomers bonded together).
Embodiment 4. The contrast agent of any of the preceding embodiments, wherein R¹ is a C₁-C₆₀ hydrocarbyl, a C₁₀-C₆₀ hydrocarbyl, a C₂₀-C₆₀ hydrocarbyl, or a C₄₀-C₆₀ hydrocarbyl.
Embodiment 5. The contrast agent of any of the preceding embodiments, wherein the amine substituent of R¹ is covalently bonded to the carbon atom of the carbonyl moiety to which “R¹” is bonded.
Embodiment 6. The contrast agent of any of the preceding embodiments, wherein (i) the amine substituent of R¹ is a secondary amine, (ii) the one or more carbon atoms substituted with one or more fluorine atoms is a trifluoromethyl group, or (iii) a combination thereof.
Embodiment 7. The contrast agent of any of the preceding embodiments, wherein R¹ is—

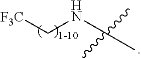

Embodiment 8. The contrast agent of any of the preceding embodiments, wherein R¹ is—

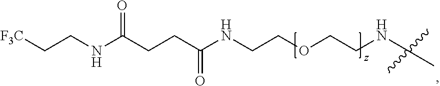

wherein z is 2 to 20, 4 to 20, 6 to 20, 8 to 20, 10 to 20, 12 to 20, 14 to 20, 16 to 20, 2 to 18, 2 to 16, 2 to 14, 2 to 12, 2 to 10, 2 to 8, 2 to 6, or 2 to 4.
Embodiment 9. The contrast agent of any of the preceding embodiments, wherein R² is—

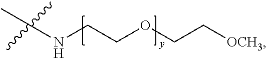

wherein v is 2 to 20, 4 to 20, 6 to 20, 8 to 20, 10 to 20, 12 to 20, 14 to 20, 16 to 20, 2 to 18, 2 to 16, 2 to 14, 2 to 12, 2 to 10, 2 to 8, 2 to 6, or 2 to 4.
Embodiment 10. The contrast agent of any of the preceding embodiments, wherein R² is a water-soluble hydrocarbyl.
Embodiment 11. The contrast agent of any of the preceding embodiments, wherein the hydrocarbyl of R² is substituted with one or more polar substituents.
Embodiment 12. The contrast agent of any of the preceding embodiments, wherein R² is a C₁-C₆₀ hydrocarbyl, a C₁₀-C₆₀ hydrocarbyl, a C₂₀-C₆₀ hydrocarbyl, or a C₄₀-C₆₀ hydrocarbyl.
Embodiment 13. The contrast agent of any of the preceding embodiments, wherein R² is a polymeric hydrocarbyl, or at least a portion of its structure is polymeric (i.e., include at least two monomers bonded together).
Embodiment 14. The contrast agent of any of the preceding embodiments, R² further comprises at least one amine, such as a primary amine, a secondary amine, a tertiary amine, or a combination thereof.
Embodiment 15. The contrast agent of Embodiment 14, wherein the amine, such as a secondary amine, is bonded to at least one monomer of polyethylene glycol, the carbon atom of the carbonyl to which R² is bonded, or a combination thereof.
Embodiment 16. A composition comprising a liquid and the contrast agent of any of Embodiments 1 to 15.
Embodiment 17. The composition of Embodiment 16, wherein the contrast agent has a solubility in the liquid of at least 0.03 mg/mL, 0.06 mg/mL, 0.1 mg/mL, 0.5 mg/mL, 1 mg/mL, 10 mg/mL, 50 mg/mL, 100 mg/mL, 150 mg/mL, 200 mg/mL, or 250 mg/mL.
Embodiment 18. The composition of Embodiment 16 or 17, wherein a concentration of the contrast agent in the liquid is 0.03 mg/mL to about 250 mg/mL, 0.06 mg/mL to about 250 mg/mL, 0.1 mg/mL to about 250 mg/mL, 0.5 mg/mL to about 250 mg/mL, 1 mg/mL to about 250 mg/mL, 10 mg/mL to about 250 mg/mL, 50 mg/mL to about 250 mg/mL, 100 mg/mL to about 250 mg/mL, 150 mg/mL to about 250 mg/mL, or 200 mg/mL to about 250 mg/mL.
Embodiment 19. The composition of any of Embodiments 16 to 18, wherein the liquid is an aqueous liquid.
Embodiment 20. A method of imaging, the method comprising administering to a patient the contrast agent or the composition of any of the preceding embodiments; and collecting an image of at least a portion of the patient with any known technique or instrument, such as magnetic resonance imaging (MRI).
Embodiment 21. The method of Embodiment 20, wherein the patient is a mammal, such as a human.
Embodiment 22. The method of Embodiment 20 or 21, wherein the contrast agent or composition is configured for oral or vascular administration.
Embodiment 23. A method of forming a contrast agent, such as a contrast agent of any of the preceding embodiments, the method comprising providing a polymer formed of at least one type of monomer, wherein the at least one type of monomer comprises a monomer comprising an anhydride; contacting the polymer with (a) a first compound comprising a primary amine and one or more carbon atoms substituted with one or more fluorine atoms, and (b) a second compound comprising polyethylene glycol substituted with at least one primary amine to form the contrast agent.
Embodiment 24. The method of Embodiment 23, wherein the polymer is a copolymer or homopolymer.
Embodiment 25. The method of Embodiment 23, wherein the polymer is poly(isobutylene-alt-maleic anhydride).
Embodiment 26. The method of Embodiment 23, wherein the polymer is of the following formula:

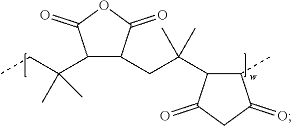

wherein w is 2 to 50, 10 to 40, 10 to 30, or 15 to 25.
Embodiment 27. The method of any of Embodiments 23 to 26, wherein the first compound is a hydrocarbon that is capable of forming an R¹ substituent of formula (I) of any of the preceding embodiments upon contacting an anhydride-containing polymer.
Embodiment 28. The method of any of Embodiments 23 to 27, wherein the first compound is—

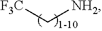

or 3,3,3-trifluoropropan-1-amine, or of the following formula:

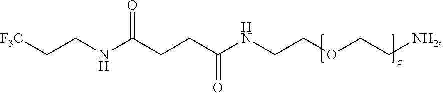

wherein z is 2 to 50, 2 to 40, 2 to 30, 2 to 20, 2 to 10, 10 to 20, 10 to 15, or 12.
Embodiment 29. The method of any of Embodiments 23 to 28, wherein the second compound is a hydrocarbon capable of forming an R² substituent of formula (I) of any of the preceding embodiments upon contacting an anhydride-containing polymer.
Embodiment 30. The method of any of Embodiments 23 to 29, wherein the second compound is of the following formula:

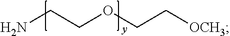

wherein y is 2 to 50, 2 to 40, 2 to 30, 2 to 20, 10 to 20, or 13 to 17.
Embodiment 31. The method of any of Embodiments 23 to 30, wherein the polymer is contacted with a mole ratio of the first compound to the second compound of about 1:99 to about 99:1, about 10:90 to about 99:1, about 20:80 to about 99:1, about 30:70 to about 99:1, about 40:60 to about 99:1, about 50:50 to about 99:1, about 60:40 to about 99:1, about 70:30 to about 99:1, about 80:20 to about 99:1, about 1:99 to about 90:10, about 10:90 to about 90:10, about 20:80 to about 90:10, about 30:70 to about 90:10, about 40:60 to about 90:10, about 50:50 to about 90:10, about 60:40 to about 90:10, about 70:30 to about 90:10, about 80:20 to about 90:10, about 10:90 to about 50:50, or about 30:70 to about 50:50.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

### Example 1—Fluorinated Polymer Design

In this example, the approach used for addressing various issues relied on an addition reaction to simultaneously introduce fluorinated moieties and hydrophilic PEG blocks onto a low molecular weight polymaleic anhydride copolymer, with a random arrangement of the fluorinated moieties and hydrophilic PEG blocks along the chain.

More precisely, two sets of fluorinated amine-R nucleophiles were used. The first was the commercially available 3,3,3-trifluoropropylamine (TFPA). The second was synthesized by inserting a tunable size, bis-reactive polyethylene glycol segment (i.e., a PEG linker/arm) between the primary amine and the CF₃ label.

An addition reaction was relied upon to install combinations of either CF₃ and PEG-OMe moieties, or PEG-CF₃ and PEG-OMe blocks laterally along the polymer backbone. This strategy achieved controlled stoichiometry and random arrangement of the CF₃ groups along the polymer chain.

Extracted were estimates of T₂ relaxation for polymers with different PEG arm-lengths between the backbone and the fluorine moieties, which confirmed that the PEGylated linkers provided substantially increased ¹⁹F resonance signal while narrowing the full width at half maximum (FWHM) peak profiles.

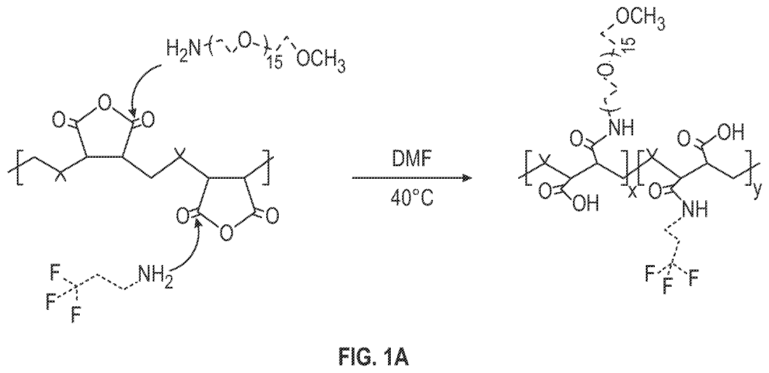

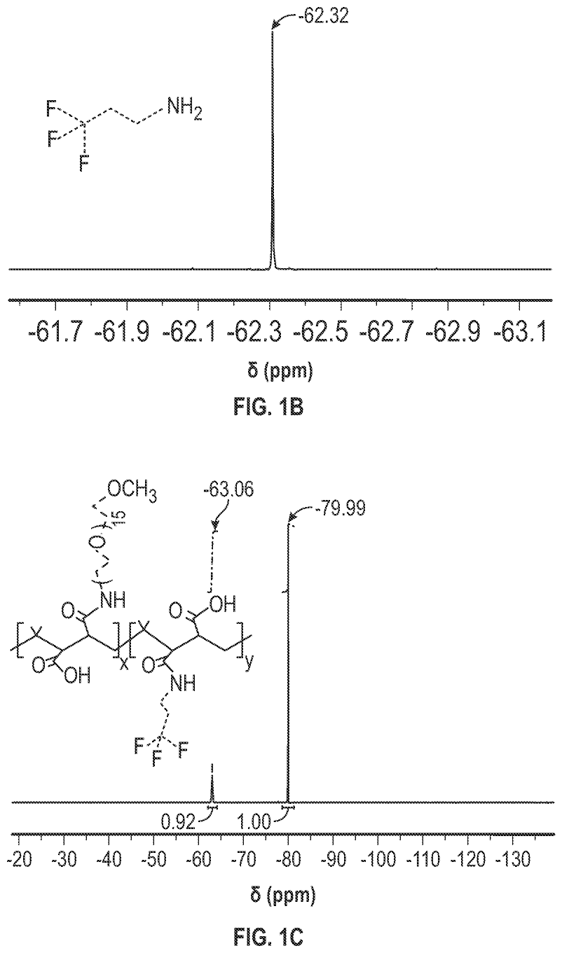

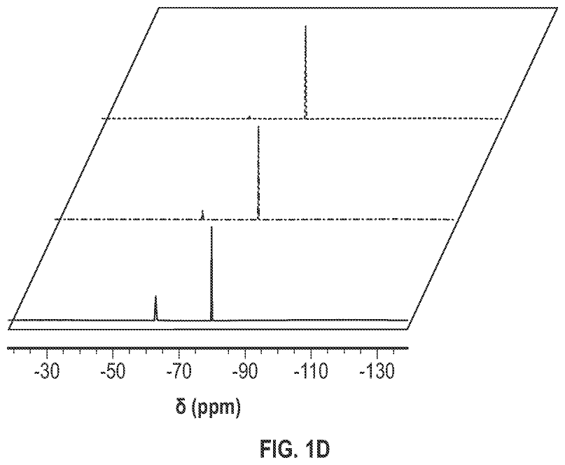

FIG. 1A depicts a schematic representation of the nucleophilic addition reaction used in this example to prepare the first set of fluorinated-polymer, PEG-OMe-PIMA-CF₃. Synthesis of the CA was realized by reacting PIMA with a mixture of commercially available TFPA and an amine-modified PEG chain (amine-PEG₇₅₀-OMe), prepared following the steps reported in previous protocols (Mei, B. C. et al. Nat Protoc 2009, 4, 412-423).

This synthetic approach allowed tuning of the polymer stoichiometry by adjusting the molar ratio(s) of the precursors with respect to the number of monomers in a PIMA chain (Wang, W. et al. J. Am. Chem. Soc. 2015, 137, 14158-14172). Here, the ¹H NMR spectra acquired using solutions in DMSO show well-defined signatures at 3.24 and 0.95 ppm ascribed to the terminal methoxy protons of PEG₇₅₀-OMe blocks and methyl protons of the PIMA backbone, respectively.

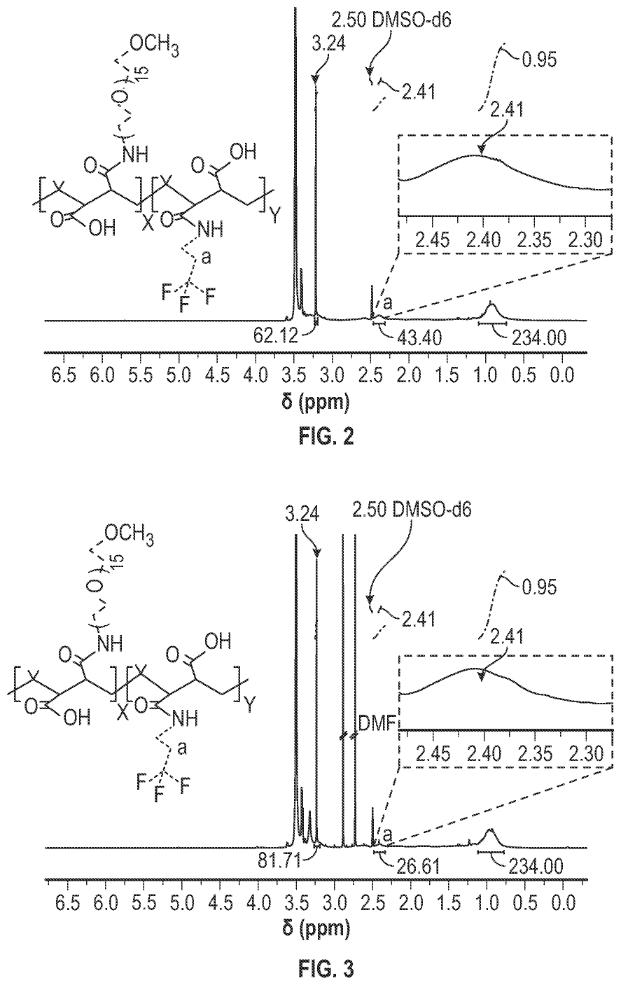

FIG. 2 depicts a ¹H NMR spectrum of PEG-OMe(50%)-PIMA-CF₃(50%) measured in DMSO-d₆. The stoichiometry was estimated by comparing the integration of the peaks ascribed to the terminal —OMe groups (at ˜3.24 ppm) and methyl groups in the PIMA backbone (at ˜0.95 ppm).

FIG. 3 depicts a ¹H NMR spectrum of PEG-OMe(70%)-PIMA-CF₃(30%) measured in DMSO-d₆. The stoichiometry was estimated by comparing the integration of the peaks ascribed to the terminal —OMe groups (at ˜3.24 ppm) and methyl groups in the PIMA backbone (at ˜0.95 ppm).

FIG. 1A is a schematic representation of nucleophilic addition reaction used to prepare the multi-functional polymer ligand PEG-OMe-PIMA-CF₃ of this example. FIG. 1B is a ¹⁹F NMR spectrum of 3,3,3-trifluoropropylamine collected in DMSO-d₆. Also, a ¹H NMR spectrum of 3,3,3-trifluoropropylamine (TFPA) was measured in DMSO-d₆. The signatures ascribed to the methylene protons (a, b) and the amine protons (c) were identified.

FIG. 1C is a ¹⁹F NMR spectrum of PEG-OMe-PIMA-CF₃(30%) collected in DMSO-d₆. The two peaks at ˜−63 and ˜−80 ppm designate CF₃ groups on the polymer and hexafluoroacetone standard respectively. FIG. 1D is a stacked ¹H NMR spectra of PEG-OMe-PIMA-CF₃(10%), PEG-OMe-PIMA-CF₃(30%) and PEG-OMe-PIMA-CF₃(50%) in the presence of HFA standard measured in DMSO-d₆.

These spectra also showed a broad peak at 2.41 ppm ascribed to the methylene adjacent to the CF₃ groups. Integration of the signatures at 2.41 ppm for TFPA and at 3.24 ppm for PEG-OMe were compared side-by-side to that measured for the PIMA methyl groups (at ˜0.95 ppm), yielding an estimate for polymer stoichiometry. In particular, a PEG-OMe:CF₃ molar ratio x:y˜70:30 was measured for PEG-OMe(70%)-PIMA-CF₃(30%), while x:y˜50:50 was deduced for the PEG-OMe(50%)-PIMA-CF₃(50%).

More precisely, the solution ¹H NMR data yielded ˜12 CF₃ and ˜27 PEG-OMe for the compound PEG-OMe(70%)-PIMA-CF₃(30%). Similarly, estimated were ˜20 CF₃ and ˜20 PEG-OMe groups per chain for PEG-OMe(50%)-PIMA-CF₃(50%) compound. These values were in good agreement with the nominal values expected from the starting molar amounts and assuming a near complete reaction (Wang, W. et al. J. Am. Chem. Soc. 2015, 137, 5438-5451).

¹⁹F NMR spectra collected from solutions of the fluorine precursor and the final polymer are depicted in FIG. 1B and FIG. 1C. Hexafluoroacetone trihydrate (HFA, which has a signature at ˜−80 ppm) was added to the solution (as an internal standard) at a fluorine concentration comparable to that anticipated for the CA (see examples below for additional details). The clearly defined peak at ˜−62 ppm was preserved in the product, albeit with a slight upfield shift to ˜−63 ppm, which indicated that the fluorine labels were successfully installed into the polymer structure.

Additionally, the approximate one-to-one ratio between the integrals of two peaks measured for the polymer and HFA was consistent with the nominal fluorine concentration expected for the nominal stoichiometry of the prepared polymer (see FIG. 1C). These results demonstrated that the addition reaction strategy could successfully install fluorine labels and PEG-OMe blocks along the PIMA chain, with controllable stoichiometry. Nonetheless, inspection of the data shown in FIG. 1C indicated a rather small fluorine signal was measured for this polymer compared to the internal standard.

Also tested was the effect of increasing the molar percentage of fluorine in the polymer on the measured signal. For this, three polymer compounds were prepared with three different molar fractions of CF₃, namely 10%, 30% and 50% (i.e., ˜4, 12 and 20 CF₃ per macromolecule). Then, aliquots of the polymer solutions in DMSO-d₆ (20 mg/mL) were used to collect the NMR spectra (shown in FIG. 1D). Table 1 summarizes the intensity ratios extracted from the NMR data for the three sets of solutions compared to the one extracted for the HFA standard. The measured intensity increased with higher fluorine content, as expected (0.03, 0.09 and 0.27 for CF₃ molar fractions of 10%, 30% and 50%, respectively. However, the overall NMR intensity stayed rather small for these sets of polymers. Additionally, white light images acquired from solutions containing 50 mg/mL of the polymer compounds in aqueous solutions showed signs of aggregation for the polymers with higher fluorine content (PEG-OMe(50%)-PIMA-CF₃(50%), see Table 1). This could be attributed to a reduced affinity of polymers with higher fluorine content to water media. The rather small NMR signal for these compounds was attributed to the slow dynamic segmental mobility of the CF₃ groups, due to their close proximity to the polymer backbone, a result similar to the weak peak measured for the methyl protons along the PIMA-chain (e.g., see FIG. 2 and FIG. 3) (Jin, Z. et al. Bioconjugate Chem. 2019, 30, 871-880).

Table 1 depicts ¹⁹F NMR signal-to-noise ratios (SNR) extracted from solutions of polymers (with a concentration=50 mg/ml) prepared at the indicated molar fraction of CF₃ groups with respect to the PIMA monomers. Data showed an increase in SNR that was commensurate with the molar fraction of CF₃. White-light images (50 mg/mL) also were acquired from PEG-OMe-PIMA-CF₃ solutions.

TABLE 1

| Polymer | SNR^{a} |
|---|---|
| PEG-OMe-PIMA-CF₃ (10%) | 0.03 |
| PEG-OMe-PIMA-CF₃ (30%) | 0.09 |
| PEG-OMe-PIMA-CF₃ (50%) | 0.27 |

^{a}The provided values are calculated with respect to the signal of the internal standard.

### Example 2—Optimizing the Architecture of the Polymer Contrast Agent

In this example, an attempt was made to lift the foregoing limitation and ultimately increase the signal-to-noise ratio (SNR) measured for the polymer CA, by introducing a PEG linker between the backbone and the fluorine groups. It was believed that using a PEG block as linker (instead of an alkyl one) would ultimately enhance the dynamic segmental mobility of the terminal CF₃, thus increasing the ¹⁹F peak intensity while potentially improving the polymer affinity to water. This rationale was supported by the large difference in the proton signatures emanating from the methyl groups in the terminal methoxy of the PEG-OMe blocks (at ˜3.24 ppm) compared to the one measured for the methyl groups along the backbone (at ˜0.95 ppm), as shown in FIG. 2 and FIG. 3. The peak at 3.24 ppm was larger than the one at 0.95 ppm, even though there a fewer OME-associated protons compared to the number of PIMA methyl protons (Jin, Z. et al. Bioconjugate Chem. 2019, 30, 871-880).

Tested were the effects of inserting a varying size PEG linker between the CF₃ group and the primary amine to generate a new amine-PEG-CF₃ nucleophile, instead of relying on a commercially available NH₂-alkyl-CF₃ compound. For a typical amine-PEG-CF₃, a starting bis-reactive NH₂-PEG₆₀₀-N₃ was converted into COOH-PEG₆₀₀-N₃ using succinic anhydride; the NH₂-PEG₆₀₀-N₃ was prepared following the steps detailed in previous protocols (see, e.g., Susumu, K. et al. J. Am. Chem. Soc. 2007, 129, 13987-13996; Susumu, K. et al. Nat. Protocols 2009, 4, 424-436). The terminal carboxylic acid was coupled to TFPA in the presence of 1,1′-carbonyldiimidazole (CDI), and then the azide in the resulting CF₃-PEG₆₀₀-N₃ was reduced to NH₂ using Staudinger reduction, ultimately yielding a new NH₂-PEG₆₀₀-CF₃ nucleophile. Details about the synthesis are provided herein.

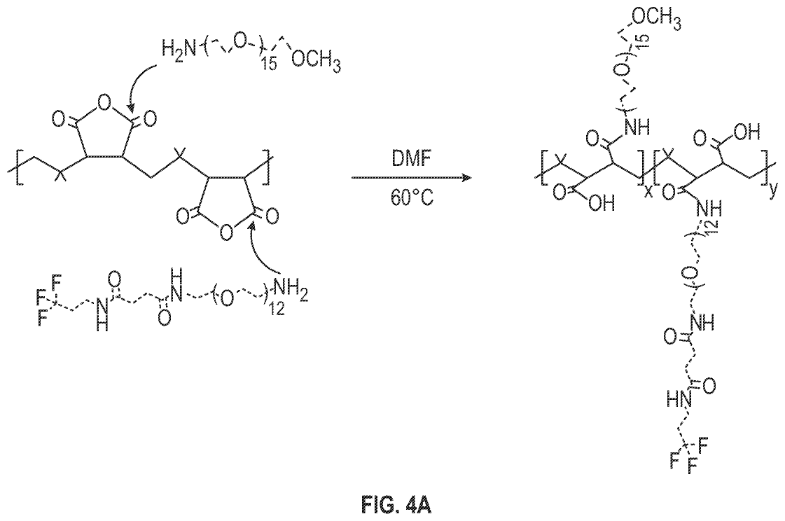

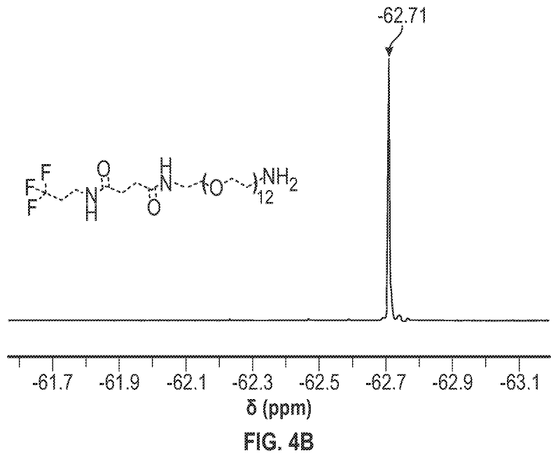

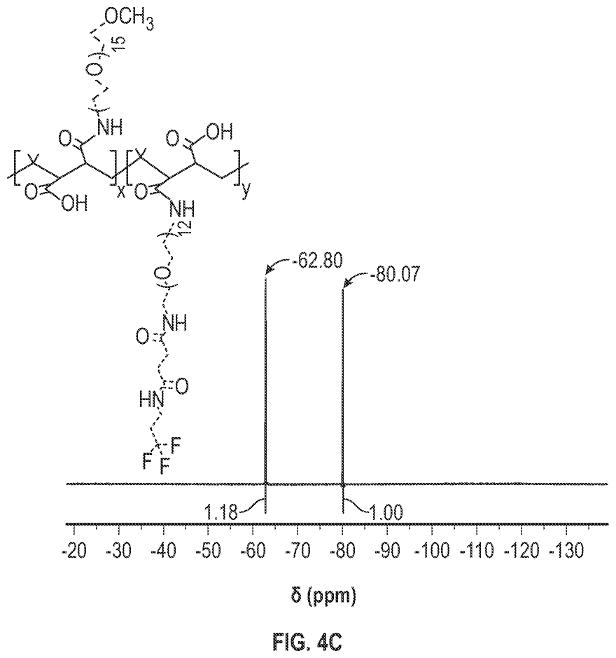

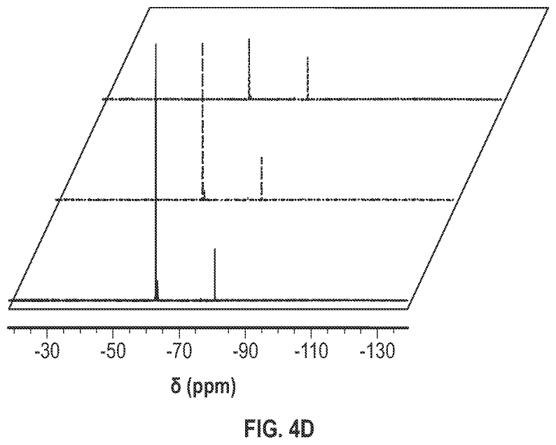

FIG. 4A schematically summarizes the nucleophilic addition reaction between PIMA and a mixture of NH₂-PEG₇₅₀-OMe and NH₂-PEG₆₀₀-CF₃. The ¹⁹F NMR spectra shown in FIG. 4B and FIG. 4C confirmed the successful insertion of the PEGylated fluorine moieties in the NH₂-PEG-CF₃ precursor and in the new PEG-OMe-PIMA-PEG₆₀₀-CF₃ polymer, with a clear well-defined peak at ˜−62 ppm for both. Furthermore, comparing the ¹⁹F peak intensities measured for NH₂-PEG₆₀₀-CF₃ and PEG-OMe-PIMA-PEG₆₀₀-CF₃ to those shown in FIG. 1C (no arm) clearly indicated a pronounced boosting in the NMR signal by nearly one order of magnitude, as a result of the change in the polymer structure, compared to the case without a PEG linker shown in FIG. 1A-FIG. 1D. This sizable improvement was attributed to the increased segmental mobility of the terminal CF₃ groups in the new polymer. Similar to what was done above for PEG-OMe-PIMA-CF₃, the effects of the fluorine content in the polymer on the measured intensity of the NMR signal were investigated. The data shown in FIG. 4D indicated that all the three sets of polymers yielded substantially higher signals compared to those acquired from polymers without the PEGylated linker. The clarity of solutions of these compounds in water was visually inspected. It was found that despite the increase in the signal to noise ratio (SNR), the polymer solutions with the highest fluorine content (PEG-OMe(50%)-PIMA-PEG₆₀₀-CF₃(50%) still showed a slightly cloudy appearance under white light exposure.

FIG. 4A is a schematic representation of the addition reaction used to prepare PEG-OME-PIMA-PEG₆₀₀-CF₃. FIG. 4B is a ¹⁹F NMR spectrum of NH₂-PEG₆₀₀-CF₃ collected in DMSO-d₆. FIG. 4C is a ¹⁹F NMR spectrum of PEG-OME-PIMA-PEG₆₀₀-CF₃ collected in DMSO-d₆. The spectrum in panel C shows two peaks at ˜−62.8 ppm and ˜−80 ppm which are ascribed to the CF₃ groups in the polymer and hexafluoroacetone standard, respectively. FIG. 4D depicts a stacked ¹⁹F NMR spectra of PEG-OMe-PIMA-PEG₆₀₀-CF₃(10%), PEG-OMe-PIMA-PEG₆₀₀-CF₃(30%), PEG-OMe-PIMA-PEG₆₀₀-CF₃(50%), measured in DMSO-d₆ in the presence of HFA.

It implied that although the PEG linker improved affinity to water, potential small aggregates could still form at high concentrations (see Table 2). Also assessed was how the presence of the PEG₆₀₀ arm affected the FWHM and T₂ relaxation time. A side-by-side comparison of spectra measured for the polymer CAs with and without the PEG₆₀₀ linker was collected. Specifically, a ¹⁹F NMR spectrum of PEG-OMe-PIMA-CF₃ and PEG-OMe-PIMA-PEG₆₀₀-CF₃ were collected in DMSO-d₆. Both polymers had PEG-OMe(50%), while the other 50% was made of CF₃ or PEG₆₀₀-CF₃. The corresponding FWHM values were ˜70.6 and 9.3 Hz, respectively. Data clearly showed that insertion of the PEG linker increased the T₂ time and concomitantly reduced the peak FWHM from ˜70.6 to ˜9.3 Hz; these two parameters are related by:

FWHM=1/(πT₂) (1)

A long relaxation time indicated higher segmental mobility, which can contribute to acquiring higher MRI signals. It should be noted that the FWHM extracted from the data using equation (1) usually differed from the experimental value, likely due to the fact that heterogeneous resonances also affected the linewidth (De Roo, J. et al. Chem. Mater. 2018, 30, 5485-5492).

Table 2 depicts ¹⁹F NMR signal-to-noise ratio acquired for PEG-OMe-PIMA-PEG₆₀₀-CF₃ with varying molar fraction of fluorinated moieties (10%, 30% and 50%). White light images (50 mg/mL) were acquired from the same solutions used to extract SNR values.

TABLE 2

| Polymer | SNR^{a} |
|---|---|
| PEG-OMe-PIMA-PEG₆₀₀-CF₃ (10%) | 1.38 |
| PEG-OMe-PIMA-PEG₆₀₀-CF₃ (30%) | 4.10 |
| PEG-OMe-PIMA-PEG₆₀₀-CF₃ (50%) | 5.75 |

^{a}The SNR values refer to the signal reported with respect to the internal standard.

Also evaluated were the effects of adjusting the PEG linker on the measured NMR signal. For this, the tests focused on the polymer prepared with a fixed fluorine content (i.e., PEG-OMe(70%)-PIMA-PEG₆₀₀-CF₃(30%), or 12 CF₃ groups). In particular, it was investigated how changing the PEG linker using PEG₁₅₀, PEG₄₀₀, PEG₆₀₀, and PEG₁₀₀₀ blocks would affect the T₂ relaxation time and the FWHM of the NMR signature. NH₂-PEG-CF₃ nucleophiles with these PEG blocks were synthesized and purified following previous protocols (Susumu, K. et al. J. Am. Chem. Soc. 2007, 129, 13987-13996). ¹H and ¹⁹F NMR spectra were acquired from solutions of the various polymers. The transverse relaxation time T₂ was measured from plots of the intensity v. echo time for solutions of the various polymers in DMSO-d₆. The resulting intensity v. time profiles were fitted using a first-exponential decay function:

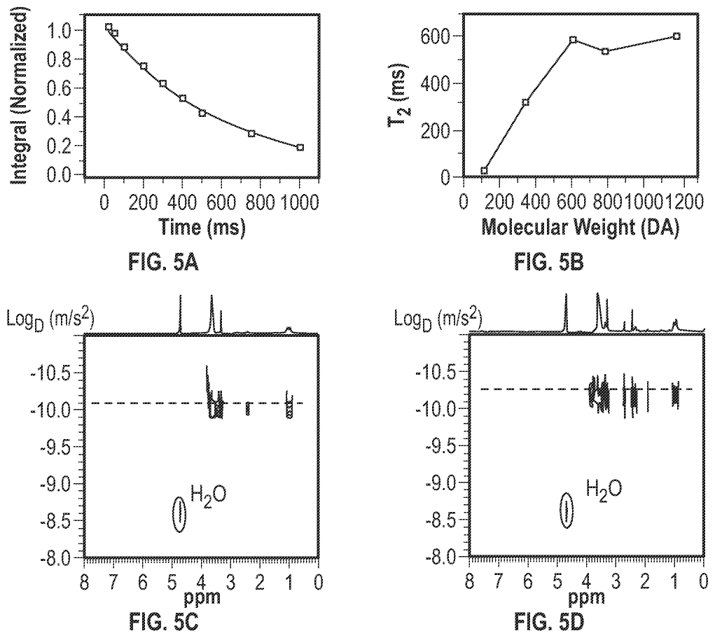

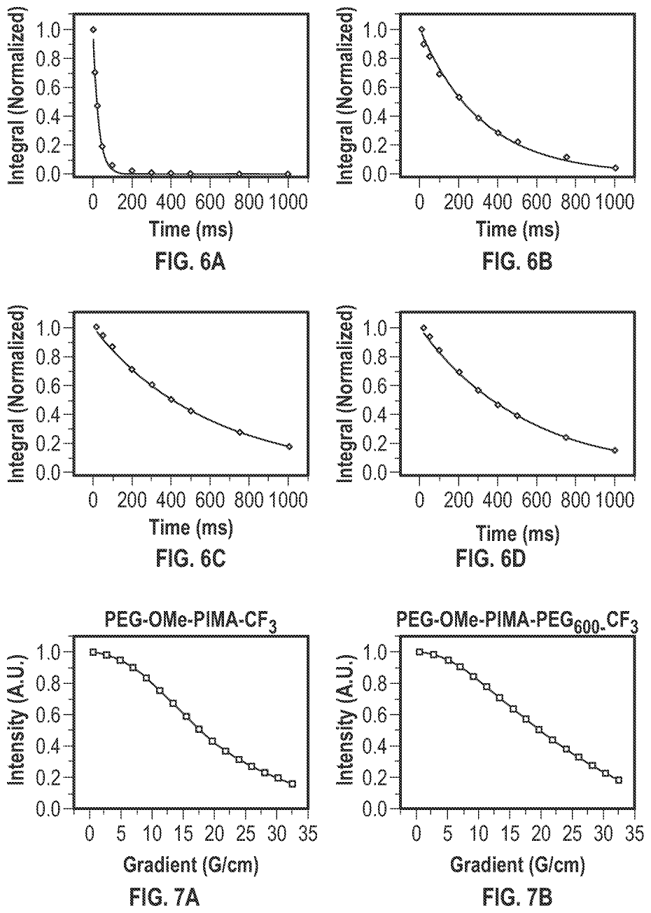

I_{Norm}=exp(−t/T₂) (2)

wherein I_{Norm} designates the normalized integrated intensity with respect to the value at t=0, while T₂ is the transverse relaxation time. T₂ values ranging from 29 ms to 596 ms were extracted from fitting the data for the various PEG linkers, as shown in FIG. 5A and FIG. 6A-FIG. 6D. A plot of T₂ vs the NH₂-PEG-CF₃ molecular weight showed that there was a correlation between the relaxation time and the molecular weight of the linker (see FIG. 5B). Inspection of the experimental data indicated that despite the limited range of PEG linkers tested, there were two trends. T₂ followed a linear progression with the molecular weight from 113 to 607 Da but saturation occurred for larger molecular weights.

FIG. 6A-FIG. 6D depict plots a normalized area of the spectra v. different echo times collected for the contrast agents. The data are fitted to a first-exponential decay function. The decay constants (T₂) are 29, 316, 581, and 533 ms for PEG-OMe-PIMA-CF₃ (FIG. 6A), PEGO-Me-PIMA-PEG₁₅₀-CF₃ (FIG. 6B), PEG-OMe-PIMA-PEG₄₀₀-CF₃ (FIG. 6C), and PEG-OMe-PIMA-PEG₆₀₀-CF₃ (FIG. 6D), respectively.

### Example 3—Characterizing the Polymer Hydrodynamic Dimensions

After confirming the capacity to increase the SNR of the CAs via structural modification, whether or not the use of such design would sizably increase the overall size of the polymer macromolecules was investigated in this example. For this, diffusion-ordered NMR spectroscopy (DOSY-NMR) was performed to characterize the hydrodynamic size of two representative polymer compounds: PEG-OMe-PIMA-CF₃ and PEG-OMe-PIMA-PEG₆₀₀-CF₃. DOSY-NMR is a non-invasive technique that can characterize the Brownian diffusion properties of small colloids and macromolecules (Groves, P., Polym. Chem. 2017, 8, 6700-6708). DOSY tracks the decay (or attenuation) of the echo NMR signal intensity as a function of the magnetic field gradient, G_{z}, which can be described by Stejskal-Tanner:

$$I = I ( 0 ) {e}^{- D {γ}^{2} {G}_{z}^{2} {δ}^{2} ( Δ - \frac{δ}{3} )} \qquad ( 3 )$$

wherein, I designates the echo intensity measured by the detector at the end of the pulse sequence, γ designates the gyromagnetic ratio of the nuclei, G_{z} is the gradient pulse strength along the z-axis, δ is the gradient pulse duration, Δ is the period separating two gradient pulses, and D is the diffusion coefficient.

FIG. 5A depicts normalized integrated intensity v. echo times collected for PEG-OMe-PIMA-PEG₁₀₀₀-CF₃. Data were fitted to an exponential decay function, yielding a decay constant (T₂) of 596 ms. FIG. 5B depicts T₂ relaxation time v. molecular weight of the fluorinated precursors. FIG. 5C and FIG. 5D depict DOSY spectra of ˜10 mg/mL PEG-OMe-PIMA-CF₃ (FIG. 5C) and PEG-OMe-PIMA-PEG₆₀₀-CF₃ (FIG. 5D) measured in D₂O. FIG. 5C and FIG. 5D represent the 2-D ¹H DOSY contour spectra measured for the PEG-OMe-PIMA-CF₃ and PEG-OMe-PIMA-PEG₆₀₀-CF₃ polymers in D₂O solutions. The corresponding intensity v. G_{z} profiles are provided in FIG. 7A and FIG. 7B. FIG. 7A and FIG. 7B depict 2-D DOSY NMR spectra of intensity v. magnetic field gradient, collected for PEG-OMe-PIMA-CF₃ (FIG. 7A) and PEG-OMe-PIMA-PEG₆₀₀-CF₃ (FIG. 7B) samples in D₂O. A concentration of 10 mg/mL was used for the experiments.

Both contour spectra showed slower diffusion coefficient signals ascribed to the polymer protons along with a faster diffusion coefficient signal emanating from the hydrogenated solvent impurities. The diffusion coefficients extracted for the two polymers were 7.21×10⁻¹¹ and 6.09×10⁻¹¹ m²/s for PEG-OMe-PIMA-CF₃ and PEG-OMe-PIMA-PEG₆₀₀-CF₃, respectively. By combining these values with the Stokes-Einstein equation:

$${R}_{H} = \frac{{k}_{B} T}{6 π η D} \qquad ( 4 )$$

wherein k_{B} is the Boltzmann constant, T is the temperature, η is the viscosity of the medium, the hydrodynamic radii (R_{H}) of the dispersed polymers were extracted: R_{H}˜3.6 and 4.0 nm for the PEG-OMe-PIMA-CF₃ and PEG-OMe-PIMA-PEG₆₀₀-CF₃, respectively. This result suggested that inserting the PEGylated linker did not drastically affect the size of the CA. Such small size bodes well for use in biological studies. In view of the data, it was concluded that the polymer prepared using a fluorine precursor with a PEGylated linker of ˜400-600 Da (i.e., NH₂-PEG₄₀₀₋₆₀₀-CF₃) had potentially yielded a promising CA. Inspecting aqueous solutions of PEG-OMe-PIMA-PEG₆₀₀-CF₃ with different concentrations essentially indicated that these CAs were fully dissolved at concentrations up to ˜50 mg/mL. White-light images of PEG-OMe-PIMA-PEG₆₀₀-CF₃ solutions were taken in DI water. The concentrations of the vials were 5, 10, 20, 25, 50, 100, 120, 140 and 160 mg/mL.

### Example 4—Phantom MR Imaging Using the Polymer Contrast Agent

The tests of this example assessed the capacity of ¹⁹F-rich polymers to provide phantom bright images in water media. Samples containing PEG-OMe-PIMA-CF₃ or PEG-OMe-PIMA-PEG₆₀₀-CF₃ were dissolved in PB buffer at varying concentrations (50, 100, 150 and 200 mg/mL) and imaged using a 900 MHz (21.1 T) MR spectrometer. First, high resolution ¹⁹F gradient-recalled echo (GRE) images were acquired to clearly demonstrate that each sample in 5-mm NMR tubes yielded high signal intensity, as evidenced by a high (0.5×0.5×1.0 mm) resolution GRE image acquired over 4 h 19 min with TE=0.846, TR=250 ms and 36 averages, which demonstrated distinct signal intensity differences between PEG-OMe-PIMA-PEG₆₀₀-CF₃ and PEG-OMe-PIMA-CF₃.

As expected, both polymer solutions exhibited increased signal intensity with increasing concentration. After confirming that the signal was distinguishable for all samples, the next step was to quantify the impact of introducing a PEGylated arm between the backbone and the fluorinated groups, in addition to the effect of varying the polymer concentration.

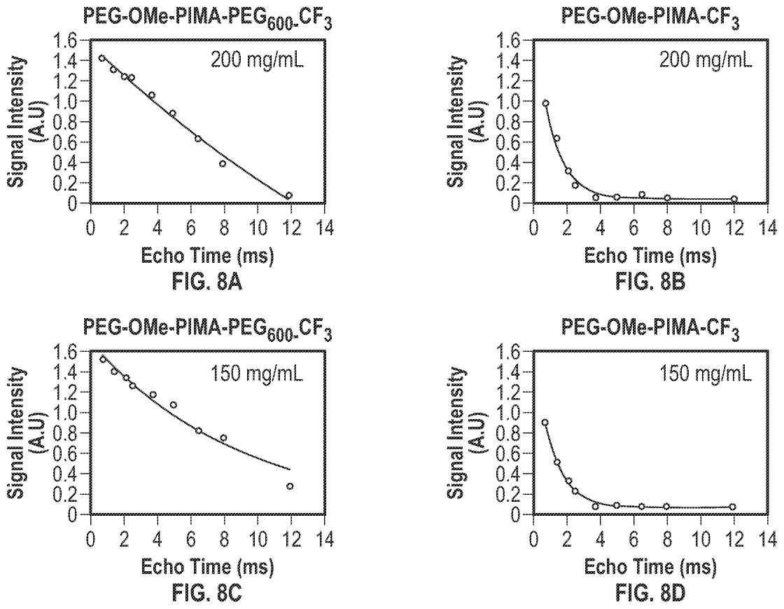

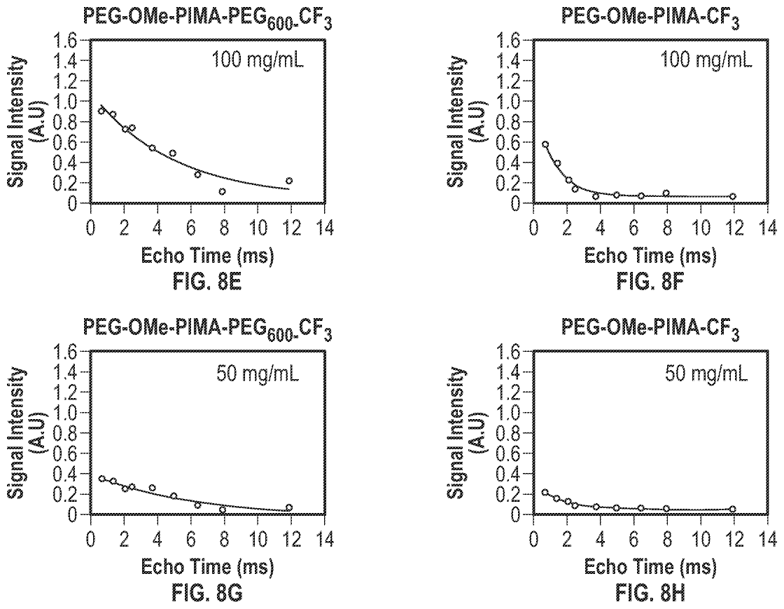

The effective T₂, or T₂*, for both polymers were determined using a series of GRE images with varying echo times, TE, (1.4 to 12.0 ms) and constant recovery time, TR. The average signal intensity within a region of each sample was plotted according to echo time, and T₂* was then extracted for each polymer and each concentration by fitting the data to a first exponential decay, as shown in FIG. 8A-FIG. 8H.

(A-H) Signal intensities (A.U.) v. echo times (ms) collected for PEG-OMe-PIMA-PEG₆₀₀-CF₃ (left) and PEG-OMe-PIMA-CF₃ (right). T₂* values were extracted from a series of GRE images with echo times 1.4, 2.1, 2.5, 3.75, 5.0, 6.5, 8.0, 12.0 ms and recovery time 100 ms, 16 averages and (1.0 mm)³ resolution. The data were fitted to a first exponential decay. The extracted T₂* values were 27.7, 9.01, 4.97, 6.21 ms for PEG-OMe-PIMA-PEG₆₀₀-CF₃ and 1.09, 1.11, 1.16, 1.60 ms for PEG-OMe-PIMA-CF₃ at 200, 150, 100, and 50 mg/mL concentrations, respectively.

Clearly, the introduction of the PEGylated arm greatly increased T₂*. T₂* also increased with increasing concentration: 6.21 v. 1.6 ms at 50 mg/mL and 27.8 v. 1.09 ms at 200 mg/mL for solutions of PEG-OMe-PIMA-PEG₆₀₀-CF₃ and PEG-OMe-PIMA-CF₃ samples, respectively. Representative T₂ weighted images clearly showed that PEG-OMe-PIMA-PEG₆₀₀-CF₃ exhibited a higher signal intensity as compared to PEG-OMe-PIMA-CF₃.

Images were collected for echo times 1.4, 2.5 ms, and 5.0 ms. Images were acquired from solutions of PEG-OMe-PIMA-PEG₆₀₀-CF₃ (50, 100, 150, 200 mg/mL), while others were PEG-OMe-PIMA-CF₃ (200, 150, 100, 50 mg/mL). Plots of the signal v. echo time for the PEG-OMe-PIMA-PEG₆₀₀-CF₃ and PEG-OMe-PIMA-CF₃ are shown in FIG. 8A-FIG. 8H.

The T₂* enhancement resulting from the addition of the PEG linker implied an increased segmental mobility of ¹⁹F groups in the system.⁵⁵ It should be noted that measurements performed in biologically compatible buffer systems (i.e. phosphate buffer), as done in this example, decreased relaxation time, specifically T₂. This, and the inherent differences between T₂ and T₂*, likely explained the reduction in values obtained between the direct NMR measurements conducted in DMSO and MRI measurements described here. Nevertheless, T₂ and T₂* were heavily influenced by the local motion exhibited by the ¹⁹F nuclei, and thus increased mobility generated by the addition of a PEG linker benefited the system by increasing spin-spin relaxation.

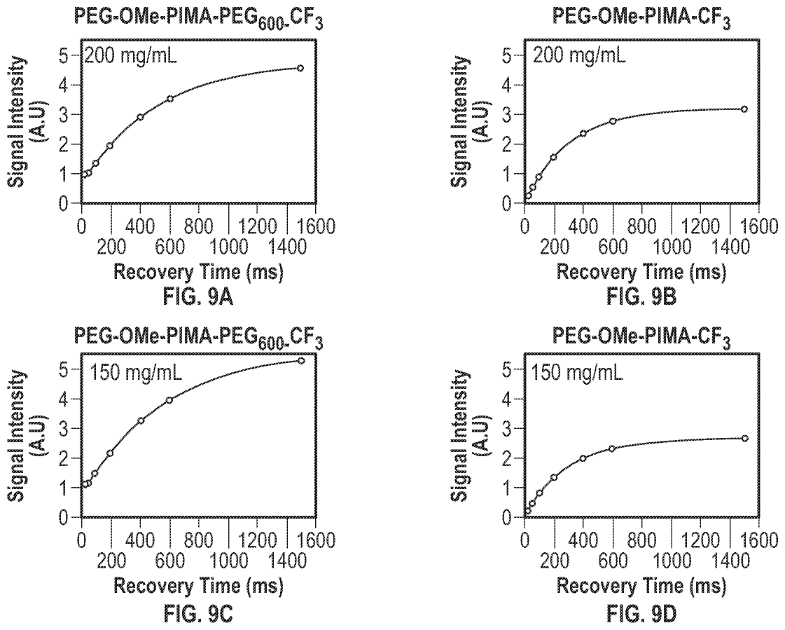

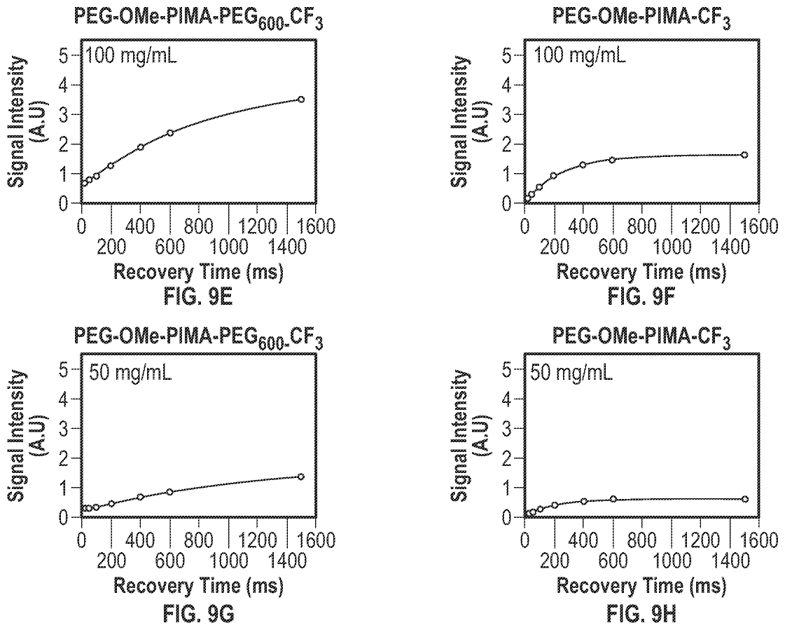

In addition to the impact on T₂*, also assessed was the effect of introducing the PEG linker on the longitudinal time T₁ using a saturation recovery experiment. T₁ values were derived from a series of GRE images with varying recovery times, 25 to 1500 ms, and constant echo time Similar to the process described above for T₂*, T₁ values were extracted from the signal intensity plotted according to recovery time by fitting the data to an exponential rise to maximum, as shown in FIG. 9A-FIG. 9H. FIG. 9A-FIG. 9H depict signal intensity (A.U.) v. recovery time (ms) collected for PEG-OMe-PIMA-PEG₆₀₀-CF₃ and PEG-OMe-PIMA-CF₃. T₁ values were extracted from a series of GRE images with recovery times 25, 50, 100, 200, 400, 600, 1500 ms and echo time 0.700 ms, 16 averages and (1.0 mm)³ resolution. Acquisition times ranged from 5 minutes 45 seconds to 7 hours 40 minutes. Extracted T₁ values were 516, 572, 853, 1300 ms for PEG-OMe-PIMA-PEG₆₀₀-CF₃ and 307, 287, 247 and 186 ms for PEG-OMe-PIMA-CF₃ for concentrations 200, 150, 100, 50 mg/mL.

For equivalent concentrations, T₁ remained high for the system containing the PEGylated linker as compared to the one without that linker. Comparing the evolution of T₁ with polymer concentration, the data indicated that T₁ decreased with increasing concentration for PEG-OMe-PIMA-PEG₆₀₀-CF₃, but it increased with increasing concentration for PEG-OMe-PIMA-CF₃. This difference could be attributed to possible aggregation or micelle formation of the polymer in the absence of the PEG linker. Comparing both systems, the enhanced water solubility in combination with a longer T₂* and a sufficiently short T₁ made the PEG-OMe-PIMA-PEG₆₀₀-CF₃ system of this example a more suitable CA in at least certain biological applications.

Phantom images were collected for recovery times 25 ms, 200 ms, and 1500 ms. In each panel, some images were acquired from solutions of PEG-OMe-PIMA-PEG₆₀₀-CF₃ (50, 100, 150, 200 mg/mL) while others were from solutions of PEG-OMe-PIMA-CF₃ (200, 150, 100, 50 mg/mL). Plots of the signal v. echo time for the PEG-OMe-PIMA-PEG₆₀₀-CF₃ and PEG-OMe-PIMA-CF₃ are shown herein.

To further demonstrate the imaging potential of this novel CA, ¹⁹F images were acquired under short acquisition time while suspended in phosphate saline buffer to mimic biological conditions. SNR calculations using the data from the phantom images were used to compare the effects of polymer architecture and concentration were performed on a GRE image acquired over 5 minutes 45 seconds with TR=25 ms and TE=0.700 ms. A 2.5-fold increase was measured for the system with the PEG linker compared to the one without that linker at 50 mg/mL concentration; this ratio increased to a 3.45-fold at 200 mg/mL. The improved SNR for the PEG-OMe-PIMA-PEG₆₀₀-CF₃ system confirmed the results and conclusions discussed above. As a result of their enhanced mobility, the fluorine moieties in PEG-OMe-PIMA-PEG₆₀₀-CF₃ gave rise to a more intense signal in MRI, compared to the polymer with short fluorinated lateral chains. The distinct difference observed in the SNR for these two CA designs strongly supported the effectiveness of introducing PEG linker chains in the chemical design, providing both enhanced water solubility and higher signal intensity.

In should be noted that the use of rather short acquisition times to collect the data yielded images with rather modest resolution, compared to others. It was expected that the SNR and resolution would be further improved with CAs based on the same strategy, but using polymers that present higher numbers of fluorinated motifs or/and moieties that present higher numbers of fluorine atoms.

The foregoing examples describe the development of a set of water compatible ¹⁹F MRI contrast agents, by combining hydrophilic PEG blocks with fluorine-rich groups in single macromolecules, where the stoichiometric ratios of those moieties were varied, as well as the architecture of the polymer. The design relied on the nucleophilic addition reaction between amine-modified PEG-OMe and PEG-CF₃ blocks and anhydride rings along a poly(isobutylene-alt-maleic anhydride) copolymer. The polymer CAs had controllable stoichiometry with good affinity to water, exhibited long T₂ relaxation times, all while having compact hydrodynamic size. It was found that introducing a PEGylated linker between the backbone and the CF₃ groups introduced additional segmental mobility of the fluorine groups, resulting in substantial enhancement in the intensity while increasing the longitudinal relaxation, T₂. These observations suggested that the developed CAs are promising for in vitro and in vivo studies, for which biocompatibility and high MR signal are desirable, if not required.

It should be noted that the addition reaction applied to PIMA also offered the flexibility of introducing specific functionalities along the polymer chain (e.g., reactive sites, peptide sequences, fluorescent species), and multifunctional ¹⁹F CAs can be easily developed using this strategy.

### Example 5—Synthesis of PEG-OMe-PIMA-CF₃

Poly(isobutylene-alt-maleic anhydride) (0.5 g, 3.25 mmol monomer units) was dissolved in 3.5 mL of DMF using in a 50-mL round bottom flask equipped with a magnetic stirring bar. Then, 3,3,3-trifluoropropylamine (183 mg, 1.625 mmol) dissolved in DMF (500 μL) was added dropwise to the PIMA solution. The content was stirred for 30 minutes before adding NH₂-PEG-OMe (1.2 g, 1.625 mmol) dissolved in 2 mL of DMF. The reaction mixture was left stirring at 30° C. overnight, and then the solvent was evaporated under vacuum. The residue was redispersed in CHCl₃ and purified by column chromatography also using CHCl₃ as eluent, to yield the fluorinated polymer product as a yellow gel; the reaction yields was ˜87%.

### Example 6—Synthesis of PEG-OMe-PIMA-PEG-CF₃

The protocol was applied for the synthesis of the various polymer compounds where the fluorinated groups were further separated from the PIMA chain by inserting a varying size PEG block between the backbone and the fluorine moieties, namely PEG-OMe-PIMA-CF₃, PEG-OMe-PIMA-PEG₁₅₀-CF₃, PEG-OMe-PIMA-PEG₄₀₀-CF₃, PEG-OMe-PIMA-PEG₆₀₀-CF₃ and PEG-OMe-PIMA-PEG₁₀₀₀-CF₃. Described in this example is the synthesis of PEG(50%)-PIMA-PEG₆₀₀-CF₃(50%) as a representative example. Poly(isobutylene-alt-maleic anhydride) (0.5 g, 3.25 mmol monomer units) was dissolved in 3.5 mL of DMF using a 50-mL round bottom flask equipped with a magnetic stirring bar. The solution was heated to 50° C., and then NH₂-PEG₆₀₀-CF₃ (1.3 g, 1.625 mmol) dissolved in DMF (2 mL) was added to the solution. The mixture was stirred for 30 minutes, and then a solution of NH₂-PEG-OMe (1.2 g, 1.625 mmol) dissolved in 2 mL of DMF was added. The reaction was left stirring overnight, and the solvent was removed under vacuum. The residue was dissolved in CHCl₃ and purified by column chromatography using CHCl₃ as eluent, yielding the PEG-OMe-PIMA-PEG₆₀₀-CF₃ as a yellow gel; the reaction yield was ˜83%. The above protocol provides a polymer with the stoichiometry PEG(50%)-PIMA-PEG-CF₃(50%), where the percentage refers to the molar fraction of the moieties compared to the total number of monomers in the PIMA chain. To prepare the polymer compounds with other stoichiometry, the molar amounts of the precursors used during the addition reaction were adjusted accordingly. It should be noted that during the synthesis of the compound with the longest PEG bridge, PEG-OMe-PIMA-PEG₁₀₀₀-CF₃, a small amount of DMSO (˜2 mL) was used to increase the solubility of the reactants.

### Example 7—¹⁹F NMR Sample Preparation

This example describes the preparation of fluorinated-polymer samples used for collecting the ¹⁹F NMR spectra (shown, for example, in FIG. 1C and FIG. 4C). Typically, to prepare a solution of PEG-OMe-PIMA-CF₃, ˜6.3 mg (0.23 μmol) of the polymer was dissolved in 450 μL of DMSO-d₆. To that solution 10 μL (1.14 μmol) of HFA standard stock solution was added. The latter solution was prepared by dissolving 12.5 mg of hexafluoroacetone trihydrate (HFA·3H₂O) in 500 μL of DI water. A similar procedure was used to prepare the NMR solutions of PEG-OMe-PIMA-PEG-CF₃. Typically, ˜8.1 mg (0.23 μmol) of the polymer was dissolved in 450 μL of DMSO-d₆, and then 10 μL of HFA standard stock was added. Identical amounts of internal standard were added to both samples to confirm that the signal shown in the NMR spectra were attributed to comparable amount of fluorine content.

### Example 8—Materials and Additional Results

3,3,3-Trifluoropropylamine (TFPA, 97%) was purchased from SynQuest Laboratories (Alachua, FL). Triethylene glycol (TEG, 99%) and 1,1′-Carbonyldiimidazole (CDI) (97%) were purchased from Acros Organics (Fisher Scientific, Lenexa, KS). Poly(ethylene glycol) (average M_{W}=400 Da) was purchased from Tokyo Chemical Industry (Portland, OR). Poly(ethylene glycol) (average M_{W}=1000 Da), poly(ethylene glycol) (average M_{W}=600 Da), poly(ethylene glycol) methyl ether (average M_{W}=750 Da), poly(isobutylene-alt-maleic anhydride) (PIMA) (average M_{W}=6000 Da), ethylenediamine (ReagentPlus®, ≥99%), along with other chemicals and solvents were purchased from Sigma Aldrich (St Louis, MO). Ultrapure water (18 M_{W}) obtained from a Milli-Q Integral 5 system (Millipore Corp., Burlington, MA), referred to as DI water, was used in all phase transfer experiments and sample preparations. Column purification chromatography was performed using silica gel (MP Silitech 60 Å pore size, 63-200 mm particle size, purchased from Thermo Fisher Scientific, Waltham, MA). The syntheses were carried out under N₂ passed through an O₂ scrubbing tower, unless otherwise stated. Air sensitive materials were handled in an Mbraun Labmaster glovebox and standard Schlenk techniques were used when handling air-sensitive materials.

Characterization: The ¹H and ¹⁹F NMR spectra acquired for the various compounds were recorded using either a 400 MHz or a 600 MHz spectrometer (Bruker SpectroSpin, Billerica, MA). The ¹⁹F NMR data were referenced against hexafluoroacetone trihydrate (HFA). The echo time series in T₂ relaxation measurements used are 1, 2, 10, 20, 50, 100, 200, 300, 400, 500, 750 and 1000 ms. The DOSY NMR data were collected using an LED-bipolar gradients pulse sequence “ledbpgp2s” with 24 k and 16 points in t₂ and t₁, respectively. Each 2D slice resulted from signal averaging over 16 scans. Typical parameters applied for a DOSY spectrum were: gradient strength=45 G/cm, diffusion delay time=300 ms, gradient duration=3.6 ms, and relaxation delay=1.0 s. Reference deconvolution and baseline correction were applied to the final spectra.

MRI data were acquired using a 900 MHz (21.1 T) spectrometer available at the National High Magnetic Field Laboratory. This magnet was equipped with a Bruker Avance III console operating PV5.0 software (Bruker SpectroSpin, Billerica, MA) and Resonance Research, Inc. imaging gradients (Billerica, MA). A switchable ¹⁹F/¹H birdcage RF coil was used for all experiments. PEG-OMe-PIMA-PEG₆₀₀-CF₃ and PEG-OMe-PIMA-CF₃ were dissolved in water at varying concentrations (50, 100, 150, 200 mg/mL) and imaged simultaneously in separate 5 mm NMR tubes. A series of Gradient Recalled Echo (GRE) images with 16 averages and (1.0 mm)³ resolution was acquired to measure saturation recovery and relaxation decay. The resulting intensity v. time profiles were fitted to either a first exponential decay (for T₂*) or exponential rise to maximum (for T₁) in Sigma Plot 11.0 (Systat Software Inc., Chicago, IL). Specifically, T₁ values were extracted from a series of GRE images with recovery times 25, 50, 100, 200, 400, 600, 1500 ms and echo time 0.700 ms. Acquisition times ranged from 5 min 45 sec to 7 h 40 min. T₂* values were extracted from a series of GRE images with echo times 1.4, 2.1, 2.5, 3.75, 5.0, 6.5, 8.0, 12.0 ms and recovery time 100 ms resulting in 23 min scan acquisitions.

Synthesis of the Fluorine-modified Polymer Compounds: Synthesis of the multifunctional ¹⁹F-based polymer contrast agent introduced in this study relied on the nucleophilic addition reaction between a polymaleic anhydride copolymer and various amine-R nucleophiles. Before detailing the polymer preparation, provided are the synthesis steps for preparing some of the amine-PEG precursors, and detailing the synthesis of the amine-PEG-fluorine ones (see the schematic herein summarizing the various compounds used).

Precursor Synthesis: The amine-terminated poly(ethylene glycol) methyl ether (NH₂-PEG-OMe), used for promoting affinity of the polymer to water, was synthesized through azide modification of poly(ethylene glycol) methyl ether followed by amine transformation, as described in reference (Mei, B. C. et al. J. Mater. Chem. 2008, 18, 4949-4958).

The fluorine-modified precursors were prepared starting with NH₂-PEG-N₃ with varying PEG blocks (namely PEG₁₅₀, PEG₄₀₀, PEG_{600, and} PEG₁₀₀₀) These compounds were synthesized by modification of HO-PEG-OH into N₃-PEG-N₃ using a two-step reaction starting with transformation to methanesulfonate in the presence of methanesulfonyl chloride, followed by transformation into azide using sodium azide, following reported protocols (Susumu, K. et al. Nat. Protocols 2009, 4, 424-436). Provided herein is a more detailed description for fluorine-labeled precursors.

Synthesis of NH₂-PEG-CF₃ Nucleophiles: Here focus was on the synthesis of NH₂-PEG₆₀₀-CF₃. The same chemical approach has been used to synthesize NH₂-PEG₁₅₀-CF₃, NH₂-PEG₄₀₀-CF₃, NH₂-PEG₆₀₀-CF₃, and NH₂-PEG₁₀₀₀-CF₃. Three reaction steps were required (see Schematics).

1. Synthesis of COOH-PEG₆₀₀-N₃. In a 500 mL three-neck round bottom flask equipped with a stir bar, NH₂-PEG₆₀₀-N₃ (9.0 g, 13.8 mmol), succinic anhydride (2.76 g, 27.6 mmol) and triethylamine (TEA, 5.1 mL, 36 mmol) were mixed with 150 mL of DCM and stirred under N₂ atmosphere at room temperature overnight. During this period the yellowish solution became brownish, dark orange, and then eventually black. After evaporation of the solvent, the residue was redispersed in 1M HCl and washed with ethyl acetate using a separatory funnel (i.e., acid wash to remove TEA and unreacted NH₂-PEG₆₀₀-N₃). The ethyl acetate layer was discarded, then DCM was added to the aqueous phase to extract the product (100 mL, three times). The combined DCM layer was dried over anhydrous Na₂SO₄. DCM was evaporated, and the crude product was purified by silica column chromatography using CHCl₃ eluent, to give COOH-PEG-N₃ as a yellow liquid; the yield was ˜83%.

2. Synthesis of CF₃-PEG₆₀₀-N₃. COOH-PEG₆₀₀-N₃ (6.1 g, 8.85 mmol) was mixed with 1,1′-carbonyldiimidazole (CDI, 1.43 g, 8.85 mmol) and 30 mL of CHCl₃ using a 100 mL two-neck round bottom flask. The mixture was stirred at room temperature for 1˜2 h then added dropwise to a solution of 3,3,3-trifluoropropylamine (1 g, 8.85 mmol) in 10 mL of CHCl₃. The content was stirred at room temperature overnight, then 30 mL of diluted HCl (pH˜4) was added, and the mixture was transferred to a separatory funnel. The organic layer was collected and concentrated to give CF₃-PEG₆₀₀-N₃ as a yellow liquid; the yield was ˜69%.

3. Synthesis of CF₃-PEG₆₀₀-NH₂. CF₃-PEG₆₀₀-N₃ (3.0 g, 3.70 mmol) was dissolved in 50 mL of dry THF in a round bottom flask, then triphenylphosphine (1.9 g, 7.40 mmol) was added. The reaction mixture was stirred at room temperature for 30 min under nitrogen atmosphere before addition of water (0.66 mL, 37.0 mmol). The mixture was further stirred overnight and then THF was removed under vacuum. The residue was dispersed in ˜7 mL of water, then the solution was transferred to a separatory funnel. The aqueous phase was washed with ethyl acetate to remove triphenylphosphine and triphenylphosphine oxide. Then, CHCl₃ was added to extract the product from water. CHCl₃ was evaporated under vacuum, yielding NH₂-PEG₆₀₀-CF₃ as a yellow gel. The reaction yield was ˜58%.

The following schemes depict chemical routes employed to prepare the various amine-PEG-CF₃ precursors, subsequently used for the synthesis of the fluorine-containing polymers:

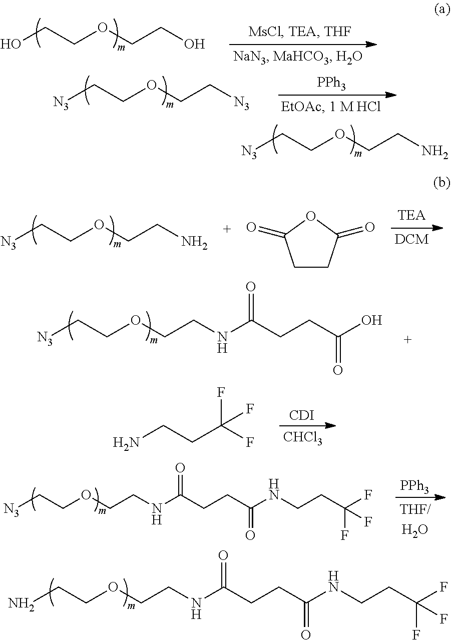

Specifically, scheme (a) shows the synthesis of the compound N₃-PEG-NH₂ which was subsequently used for the synthesis of the various CF₃ precursors, and scheme (b) shows the synthesis of amine-PEG-CF₃, which relied on the succinic anhydride transformation of the terminal amine (in NH₂-PEG-N₃) to yield in COOH-PEG-N₃, followed by CDI coupling, then azide to amine transformation of the remaining terminal azide.

1. A contrast agent of the following formula: 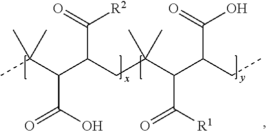
wherein a ratio of x:y is about 30:70 to about 99:1;
wherein, independently, each R¹ is a hydrocarbyl that (i) is substituted with an amine, and (ii) comprises one or more carbon atoms substituted with one or more fluorine atoms; and
wherein R² comprises polyethylene glycol.

2. The contrast agent of claim 1, wherein—
(i) R¹ is— 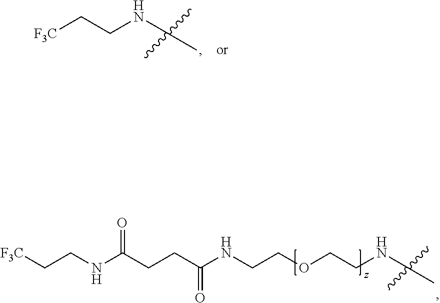
wherein z is 2 to 20; and
(ii) R² is— 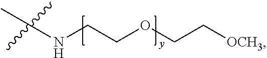
wherein v is 2 to 20.

3. The contrast agent of claim 1, wherein the ratio of x:y is about 50:50 to about 90:10.

4. The contrast agent of claim 1, wherein (i) the amine of R¹ is a secondary amine, (ii) the one or more carbon atoms substituted with one or more fluorine atoms is a trifluoromethyl group, or (iii) a combination thereof.

5. The contrast agent of claim 4, wherein R¹ is—

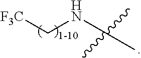

6. The contrast agent of claim 4, wherein R¹ is— 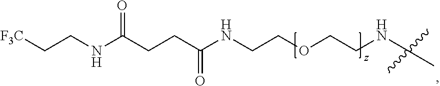
wherein z is 2 to 20.

7. The contrast agent of claim 1, wherein R² further comprises a secondary amine bonded to at least one monomer of the polyethylene glycol.

8. The contrast agent of claim 7, wherein R² is— 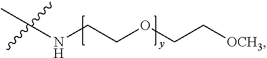
wherein v is 2 to 20.

9. A composition comprising:
a liquid; and
the contrast agent of claim 1;
wherein the contrast agent has a solubility in the liquid of at least 1 mg/mL.

10. The composition of claim 9, wherein the contrast agent has (i) a solubility in the liquid of at least 10 mg/mL, (ii) a concentration of the contrast agent in the liquid is about 10 mg/mL to about 250 mg/mL, or (iii) a combination thereof.

11. The composition of claim 10, wherein the liquid is an aqueous liquid.

12. A method of imaging, the method comprising:
administering to a patient the contrast agent of claim 1; and
collecting an image of at least a portion of the patient with magnetic resonance imaging (MRI).

13. A method of forming a contrast agent, the method comprising:
providing a polymer formed of at least one type of monomer, wherein the at least one type of monomer comprises a monomer comprising an anhydride;
contacting the polymer with (a) a first compound comprising a primary amine and one or more carbon atoms substituted with one or more fluorine atoms, and (b) a second compound comprising polyethylene glycol substituted with at least one primary amine to form the contrast agent.

14. The method of claim 13, wherein the polymer is of the following formula: 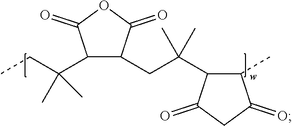
wherein w is 10 to 30.

15. The method of claim 13, wherein the one or more carbon atoms substituted with one or more fluorine atoms comprises a terminal trifluoromethyl group.

16. The method of claim 13, wherein the first compound is 3,3,3-trifluoropropan-1-amine, or 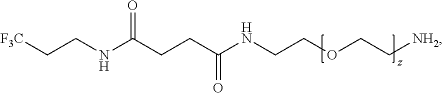
wherein z is 2 to 20.

17. The method of claim 13, wherein the second compound further comprises a terminal alkoxy moiety.

18. The method of claim 13, wherein the second compound is of the following formula: 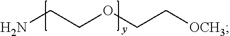
wherein y is 2 to 20.

19. The method of claim 13, wherein the polymer is contacted with a mole ratio of the first compound to the second compound of about 10:90 to about 50:50.

20. The method of claim 13, wherein the polymer is contacted with a mole ratio of the first compound to the second compound of about 30:70 to about 50:50.
